(19) United States
(12) Patent Application Publication
(10) Pub. No.: US 2024/0360075 A1
(43) Pub. Date: Oct. 31, 2024

(54) N-SUBSTITUTED C6 CYCLYL CARBOXAMIDE COMPOUNDS AND USES THEREOF

(71) Applicant: ALCON INC., Fribourg (CH)

(72) Inventors: Heeren M. Gordhan, Durham, NC (US); Mitchell A. deLong, Chapel Hill, NC (US); Jill M. Sturdivant, Chapel Hill, NC (US); David Archer Ellis, Durham, NC (US); Cynthia L. Lichorowic, Durham, NC (US)

(21) Appl. No.: 18/640,931

(22) Filed: Apr. 19, 2024

Related U.S. Application Data
(60) Provisional application No. 63/460,422, filed on Apr. 19, 2023.

(51) Int. Cl. C07C 233/60; A61K 9/00; A61K 31/16; A61K 31/165; A61K 31/167; A61K 31/175; A61K 31/192; A61K 31/27; A61K 31/277; A61K 31/337; A61K 31/341; A61K 31/343; A61K 31/351; A61K 31/382; A61K 31/397; A61K 31/40; A61K 31/415; A61K 31/425; A61K 31/4465; A61K 31/495; A61K 31/5375; A61K 31/69; A61K 47/12; A61K 47/18; A61K 47/38; C07C 231/02; C07C 233/58; C07C 233/59; C07C 233/62; C07C 233/63; C07C 235/14; C07C 235/56; C07C 237/24; C07C 271/20; C07C 271/24; C07C 279/22; C07C 315/04; C07C 317/30; C07D 205/04; C07D 207/09; C07D 209/54; C07D 211/58; C07D 213/75; C07D 213/79; C07D 231/40; C07D 239/42; C07D 263/04; C07D 263/56; C07D 265/36; C07D 275/03; C07D 277/46; C07D 295/088; C07D 295/135; C07D 295/32; C07D 305/06; C07D 305/08; C07D 305/14; C07D 307/22; C07D 307/94; C07D 309/14; C07D 311/58; C07D 335/02; C07F 5/02

(52) U.S. Cl. CPC C07C 233/60; A61K 9/0048; A61K 31/16; A61K 31/165; A61K 31/167; A61K 31/175; A61K 31/192; A61K 31/27; A61K 31/277; A61K 31/337; A61K 31/341; A61K 31/343; A61K 31/351; A61K 31/382; A61K 31/397; A61K 31/40; A61K 31/415; A61K 31/425; A61K 31/4465; A61K 31/495; A61K 31/5375; A61K 31/69; A61K 47/12; A61K 47/183; A61K 47/186; A61K 47/38; C07C 231/02; C07C 233/58; C07C 233/59; C07C 233/62; C07C 233/63; C07C 235/14; C07C 235/56; C07C 237/24; C07C 271/20; C07C 271/24; C07C 279/22; C07C 315/04; C07C 317/30; C07D 205/04; C07D 207/09; C07D 209/54; C07D 211/58; C07D 213/75; C07D 213/79; C07D 231/40; C07D 239/42; C07D 263/04; C07D 263/56; C07D 265/36; C07D 275/03; C07D 277/46; C07D 295/088; C07D 295/135; C07D 295/32; C07D 305/06; C07D 305/08; C07D 305/14; C07D 307/22; C07D 307/94; C07D 309/14; C07D 311/58; C07D 335/02; C07F 5/025; C07C 2601/02; C07C 2601/04; C07C 2601/08; C07C 2601/14; C07C 2602/18; C07C 2602/38; C07C 2602/42; C07C 2602/50

(57) ABSTRACT

Described herein are modified monoterpene TRPM8 activating compounds. In particular, provided herein are compounds that affect the function of ion channels in a cell, and are useful as therapeutic agents or with therapeutic agents. The compounds provided herein are useful in the treatment of a variety of diseases and conditions including inflammatory eye diseases such as uveitis, cardiovascular diseases, inflammatory diseases, and diseases characterized by abnormal growth, such as cancers.

### RELATED APPLICATIONS

This application claims priority from U.S. Provisional Patent Application No. 63/460,422, filed Apr. 19, 2023, the entire content of which is incorporated herein by reference.

### BACKGROUND

Transient receptor potential (TRP) channels are a group of ion channels located mostly on the plasma membrane of numerous animal cell types. TRP channels function as nonselective cation-permeable channels, and, when activated, depolarize the cell, leading to subsequent voltage-dependent ion channel activation and changes in intracellular Ca²⁺ concentrations. Their classification is based on sequence similarities and not on their common functional features. Most of these channels are grouped into two broad groups: Group 1 includes TRPC (“C” for canonical), TRPV (“V” for vanilloid), TRPVL (“VL” for vanilloid-like), TRPM (“M” for melastatin), TRPS (“S” for soromelastatin), TRPN (“N” for no mechanoreceptor potential C), and TRPA (“A” for ankyrin). Group 2 consists of TRPP (“P” for polycystic) and TRPML (“ML” for mucolipin). Other less-well categorized TRP channels exist, including yeast channels and a number of Group 1 and Group 2 channels present in non-animals. Many TRP channels mediate a variety of sensations such as pain, temperature, different kinds of tastes, pressure, and vision. Some TRP channels act as sensors of osmotic pressure, volume, stretch, and vibration. Most of the channels are activated or inhibited by signaling lipids and contribute to a family of lipid-gated ion channels. TRP channels are found in vertebrates where they are ubiquitously expressed in many cell types and tissues. Most TRP channels are composed of 6 membrane-spanning helices with intracellular N- and C-termini. Mammalian TRP channels are activated and regulated by a wide variety of stimuli and are expressed throughout the body.

TRP ligands may modulate multiple TRP channels resulting in an activity profile that includes elicitation of undesirable sensation. Thus, there is a need for selective TRP channel ligands, including selective TRP channel subfamily M (melastatin) member 8 (TRPM8), also known as the cold and menthol receptor 1 (CMR1), and TRP channel polycystin-2 (TRPP2) ligands.

### SUMMARY

Described herein are N-substituted C₆ cyclyl carboxamide compounds, which affect cellular ion transport, such as the Ca²⁺ ion, and are useful as therapeutic agents or in combination with one or more additional therapeutic agents. In some embodiments, the compounds described herein are useful: for the treatment of eye diseases including, without limitation, non-infectious uveitis, non-infectious chorioretinitis, iritis, sterile conjunctivitis, keratitis, episcleritis, dry eye diseases, meibomian gland dysfunction, allergic conjunctivitis, glaucoma, or retinal diseases; as anti-inflammatory agents; for the treatment of skin diseases; for the treatment of cardiovascular diseases; for the treatment of auto-immune disorders including, without limitation, rheumatoid arthritis, Crohn's disease, ulcerative colitis; or for the treatment of diseases characterized by abnormal growth, including, without limitation cancers, including, without limitation, prostate cancer.

### DETAILED DESCRIPTION

N-substituted C₆ cyclyl carboxamide compounds, which are selective ligands for TRPM8, TRPP2, or both over one or more other TRP channels or one or more groups of TRP channels, have been discovered.

### Definitions

While the disclosure has been described in detail and with reference to specific embodiments, it will be apparent to one skilled in the art that various changes and modifications can be made without departing from the spirit and scope of the disclosure.

Groupings of alternative elements or embodiments disclosed herein may be referred to and claimed individually or in any combination with other members of the group or other elements found herein.

Unless otherwise defined, all technical and scientific terms used herein are accorded the meaning commonly known to one with ordinary skill in the art.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, methods of chemical transformation, protecting group methodologies, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995); the entire contents of each of which are incorporated herein by reference.

Those skilled in the art will recognize, or be able to ascertain, using no more than routine experimentation, many equivalents of the specific embodiments of the invention described herein. Such equivalents are intended to be encompassed by the claims below.

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the term about refers to an error of +10%. Otherwise, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “alkyl” refers to branched, cyclic, or straight chain, or a combination thereof, saturated hydrocarbon moieties.

The term “alkylene”, as used herein, refers to a divalent alkyl group.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The term “aryl” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Representative examples of aryl include, but are not limited to, indolyl, naphthyl, phenyl, quinolinyl and tetrahydroquinolinyl.

The term “Cₑ₋ₖ” refers to a moiety comprising e to k carbon atoms, wherein e and k are integers.

The terms “composition” and “pharmaceutical composition” refer to a mixture of at least one compound described herein with a carrier or a pharmaceutically acceptable carrier, respectively. The pharmaceutical composition facilitates administration of the compound to a patient or subject. Multiple techniques of administering a composition exist including, but not limited to, intravenous, oral, nasal, rectal, intravaginal, aerosol, parenteral, buccal, sublingual, ophthalmic, pulmonary, transdermal, and topical administration.

The term “cycloalkyl” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. A cycloalkyl group can be substituted or unsubstituted.

The terms “effective amount” and “therapeutically effective amount” refer to an amount of therapeutic compound, such as a compound described herein, administered to a subject, either as a single dose or as part of a series of doses, which is effective to produce a desired therapeutic effect.

The term “halogen” or “halo” as used herein, means Cl, Br, I, or F.

The term “haloalkyl” as used herein, refers to at least one halogen atom appended to the parent molecular moiety through an alkyl group, as defined herein.

The term “heteroalkyl” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides. A heteroalkyl group can be substituted or substituted.

The term “heteroaryl” as used herein, refers to an aromatic monocyclic ring or an aromatic bicyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Representative examples of heteroaryl include, but are not limited to, indolyl, pyridinyl (including pyridin-2-yl, pyridin-3-yl, pyridin-4-yl), pyrimidinyl, thiazolyl, and quinolinyl. A heteroaryl group can be substituted or substituted.

The term “heterocycle” or “heterocyclic” as used herein, means a monocyclic heterocycle, a bicyclic heterocycle, or a tricyclic heterocycle. The monocyclic heterocycle is a three-, four-, five-, six-, seven-, or eight-membered ring containing at least one heteroatom independently selected from the group consisting of 0, N, and S. The three- or four-membered ring contains zero or one double bond, and one heteroatom selected from the group consisting of 0, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of 0, N, and S. The seven- and eight-membered rings contains zero, one, two, or three double bonds and one, two, or three heteroatoms selected from the group consisting of 0, N, and S. A heterocyclic group can be substituted or unsubstituted.

The term “pharmaceutically acceptable carrier” means a pharmaceutically acceptable material, composition, or carrier, such as a liquid filler, solid filler, stabilizer, dispersing agent, suspending agent, diluent, excipient, thickening agent, solvent, or encapsulating material, involved in carrying or transporting at least one active pharmaceutical ingredient described herein within or to the patient such that the active pharmaceutical ingredient may perform its intended function. A given carrier must be “acceptable” in the sense of being compatible with the other ingredients of a particular cream formulation, including the active pharmaceutical ingredients described herein, and not injurious to the patient. Other ingredients that may be included in the pharmaceutical creams described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Heinrich Stahl & Camille G. Wermuth (Eds.), VHCA & Wiley-VCH, 2002), the entire content of which is incorporated herein by reference. Reference to a type or class of compound herein may be understood as including pharmaceutically acceptable salts of that compound.

The term “refractory disease” refers to a disease that continues to progress during treatment with a pharmaceutical ingredient other than the active pharmaceutical ingredients provided herein, partially responds to the other treatment, or transiently responds to the other treatment. The term may be applied to each of the diseases referred to herein.

The terms “substituted” or “substitution” refers to replacement of hydrogen attached to another group with an atom or group of atoms as the replacement substituent, wherein each substituent is independently selected.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

All methods described herein can be performed in any suitable order unless otherwise indicated herein or otherwise clearly contradicted by context. The use of any and all examples, or exemplary language (e.g., “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc., at ambient temperatures.

Reference, if any, made to a patent document or other publication in this specification serves as an incorporation herein by reference of the entire content of such document or publication.

Embodiments of this disclosure are illustrative. Accordingly, the present disclosure is not limited to that precisely as shown and described.

### Compounds

N-substituted C₆ cyclyl (e.g., cyclohexyl or phenyl) carboxamide compounds are described.

In some embodiments, provided herein are compounds having the formula:

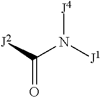

or a pharmaceutically acceptable salt thereof,
wherein
J¹ is C₁₋₁₃H₂₋₁₉N₀₋₂O₀₋₃S₀₋₁B₀₋₁F₀₋₃Cl₀₋₁ comprising 0 to 3 rings and a molecular weight of about 39.0 to about 220.3 (e.g., about 80 to about 200);
J² is

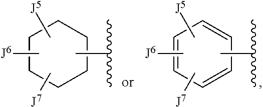

wherein
   J⁵ is halo, C₁₋₃ alkyl, C₁₋₃ haloalkyl, OH, NH₂, N(H)Ac, N(H)Boc, N(H)Fmoc, or N(H)Cbz;
   J⁶ is H; and
   J⁷ is H, propyl, ethyl-NH₂, ethyl-OH, acetyl, propenyl, propenolyl, methyloxiranyl, or methylenecyclopropanyl;
   or J⁶ and J⁷ together form a C₁₋₆ alkylenyl bridge; and
J⁴ is H, C₁₋₆ heteroalkyl, C₆₋₁₂ aryl, or C(O)—(C₁₋₃ alkylene)-(C₂₋₈ heterocycloalkyl);
or J¹ and J⁴, together with the nitrogen to which they are attached, form an unsubstituted C₂₋₈ heterocycloalkyl, or C₂₋₈ heterocycloalkyl substituted with one or two groups independently selected from C₁₋₆ alkyl or C₁₋₆ haloalkyl, wherein hetero refers to one, two, or three of, independently, N, O, or S.

In some embodiments, J¹ is C₁₋₆ alkyl-NH₂, (CH₂)₀₋₂—CN, (CH₂)₀₋₂—CCH, (CH₂)₀₋₂-Ph, (CH₂)₀₋₂-Ph-O—(C₁₋₆ alkyl), (CH₂)₀₋₂-Ph-((C₂₋₅ heterocycloalkyl)-(C₁₋₃ alkyl)), (CH₂)₀₋₂-Ph-O—(C₁₋₆ heteroalkyl), (CH₂)₀₋₂-Ph-(C₁₋₆ alkyl), (CH₂)₀₋₂-Ph-(C₁₋₆ alkyl-NH₂), (CH₂)₀₋₂-Ph-OH, (CH₂)₀₋₂-Ph-(C₁₋₆ alkyl-OH), (CH₂)₀₋₂-Ph-(C₃₋₆ cycloalkyl), (CH₂)₀₋₂-Ph-(C₂₋₈ heterocycloalkyl), or (CH₂)₀₋₂-Ph-O—(C₁₋₆ alkyl)-(C₂₋₈ heterocycloalkyl).

In some embodiments, J¹ is C₁₋₁₃H₂₋₁₉N₀₋₂O₀₋₃S₀₋₁B₀₋₁F₀₋₃Cl₀₋₁ comprising 0 to 3 rings and a molecular weight of about 80 to about 200. In some embodiments, J¹ comprises 1, 2, or 3 rings. In some embodiments, J¹ comprises a molecular weight of about 80 to about 200.

In some embodiments, J¹ comprises at least one atom selected, independently, from N, O, S, B, F, or Cl. In some embodiments, J¹ comprises at least two atoms selected, independently, from N, O, S, B, F, or Cl. In some embodiments, J¹ is not p-anisolyl.

In some embodiments,

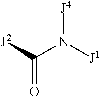

is

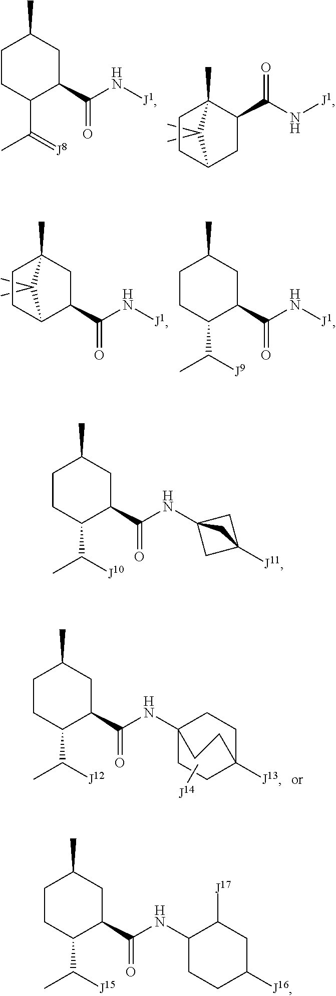

or a pharmaceutically acceptable salt thereof,
wherein
J⁸ is CH₂ or O;
J⁹ is CH₃, NH₂, or OH;
J¹⁰ is CH₃, NH₂, or OH;
J¹¹ is NH₂, CN, C₁₋₃ alkyl, F, C₁₋₃ haloalkyl, O(C₁₋₃ alkyl), (C₁₋₃ alkylenyl)CN, (C₁₋₃ alkylenyl) OH, (C₁₋₃ alkylenyl)O(C₁₋₃ alkyl), C(O)OH, (C₁₋₃ alkylenyl)C(O)OH, C(O)O(C₁₋₃ alkyl), (C₁₋₃ alkylenyl)C(O)O(C₁₋₃ alkyl), C(O)O(C₁₋₆ alkyl), tetramethyldioxaborolanyl, CO₂NH₂, C(O)N(C₁₋₃ alkyl)O(C₁₋₃ alkyl), N(H)C(O)(C₁₋₃ alkylenyl)OH, or N(H)C(O)O(C₁₋₃ alkyl);
J¹² is CH₃, NH₂, or OH;
J¹³ is O(C₁₋₃ alkyl) or C(O)O(C₁₋₃ alkyl);
J¹⁴ is H;
J¹⁵ is CH₃, NH₂, or OH;
J¹⁶ is H, O(C₁₋₃ alkyl), or O(C₁₋₃ alkylenyl)O(C₁₋₃ alkyl); and
J¹⁷ is H, or forms a C₈₋₁₀ spirocycloalkyl substituted with NH₂ or NHC(O)(C₁₋₃ alkylenyl)NH₂.

In some embodiments, J¹¹ is NH₂, CN, CH₂CH₃, F, CH₂CF₃, OCH₃, OCH₂CH₃, CH₂CN, CH₂OH, CH₂CH₂OH, CH₂OCH₃, C(O)OH, CH₂C(O)OH, C(O)OCH₃, CH₂C(O)OCH₃, C(O)OC(CH₃)₃, tetramethyldioxaborolanyl, CO₂NH₂, C(O)N(CH₃)OCH₃, N(H)C(O)CH₂OH, or N(H)C(O)OCH₃.

In some embodiments, J¹³ is OCH₃ or C(O)OCH₃.

In some embodiments, J¹⁶ is H, OCH₃, or OCH₂CH₂OCH₃.

In some embodiments, J¹⁷ is H, or forms a spirononanyl substituted with NH₂ or NHC(O)CH₂NH₂.

In some embodiments, J⁹, J¹⁰, J¹², and J¹⁵ are CH₃.

In some embodiments, J¹ is

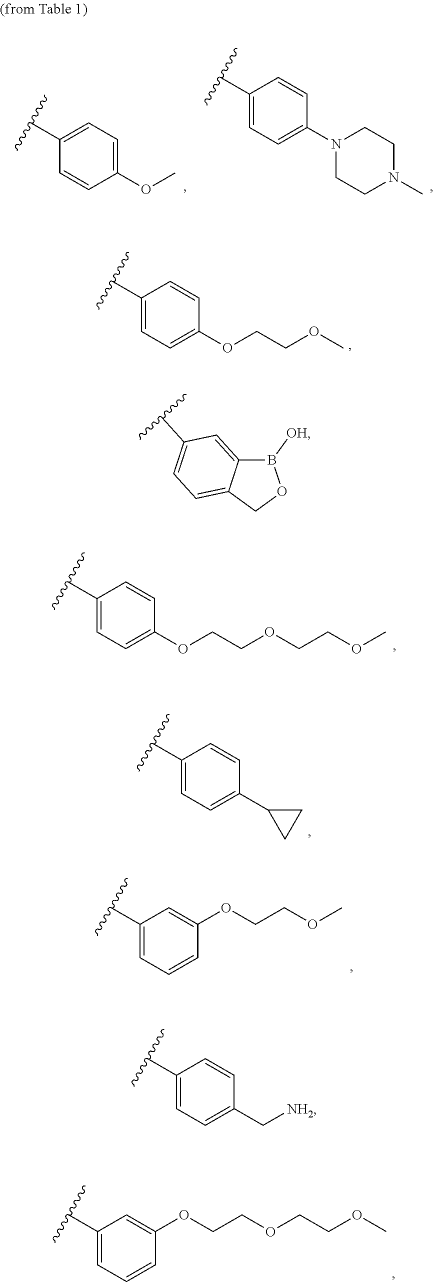

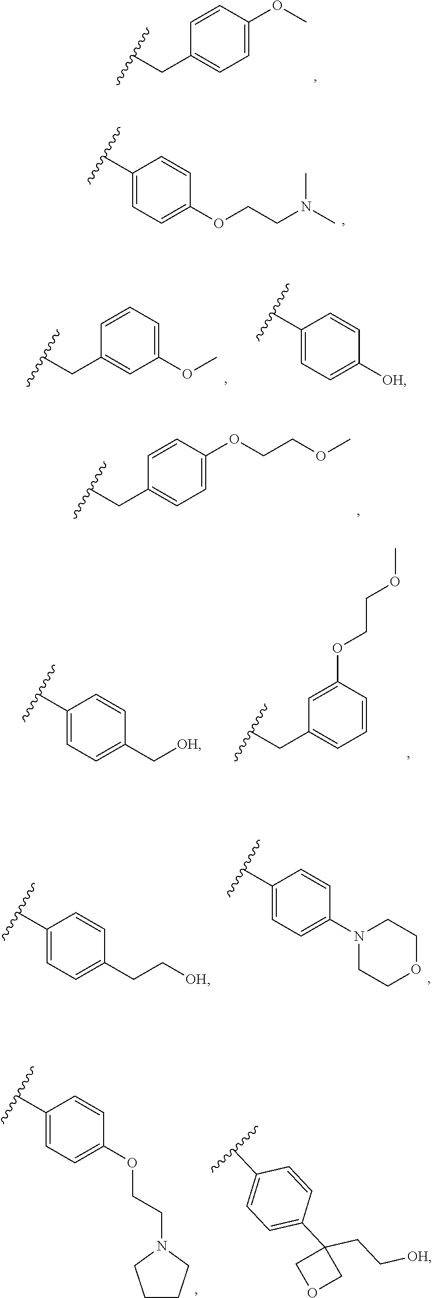

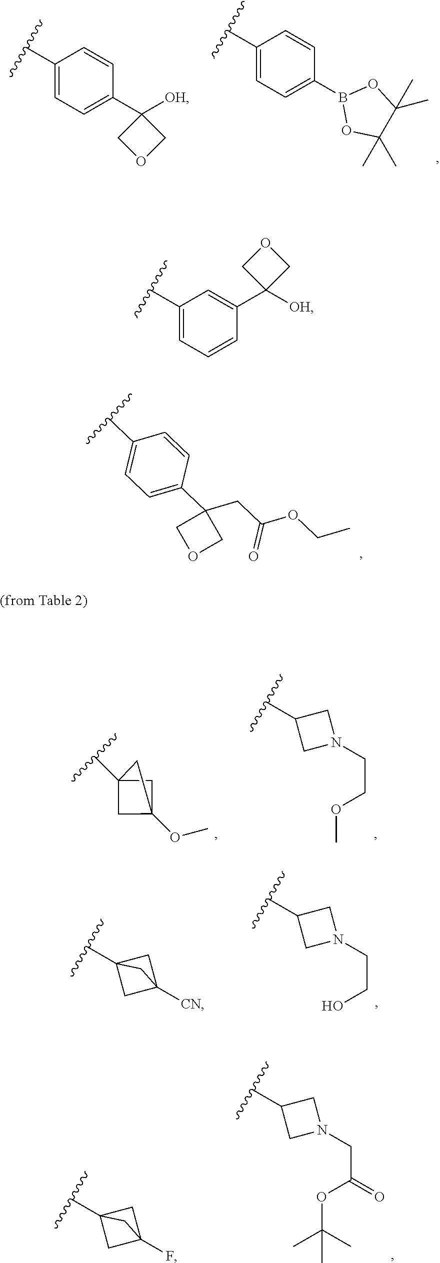

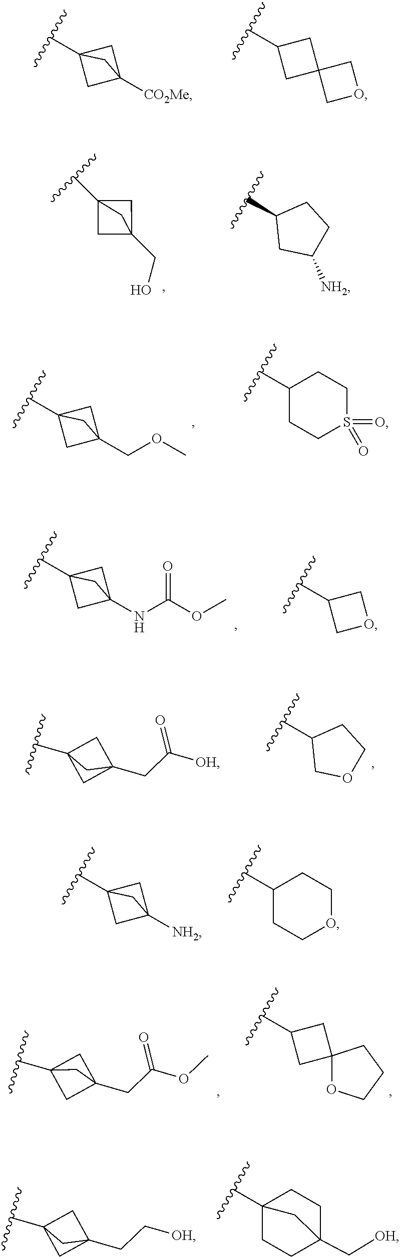

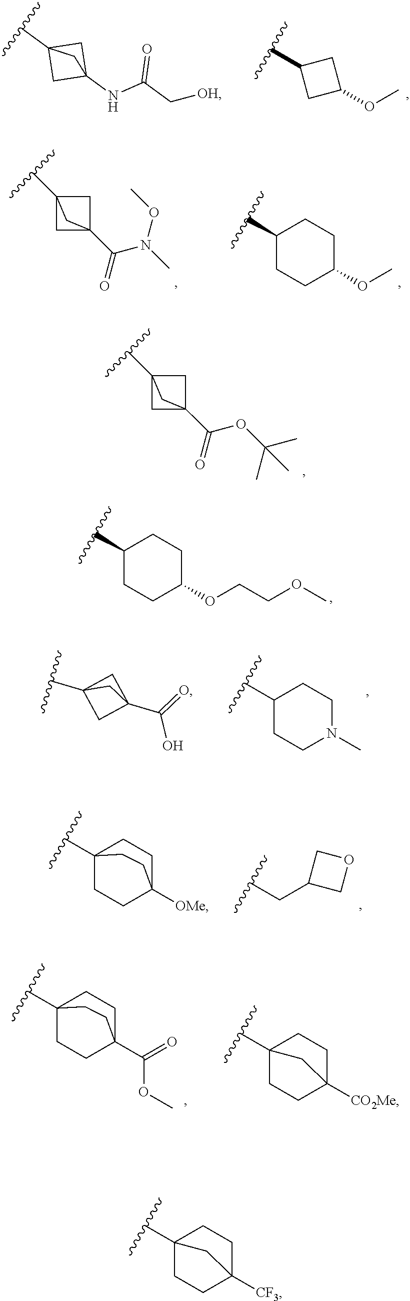

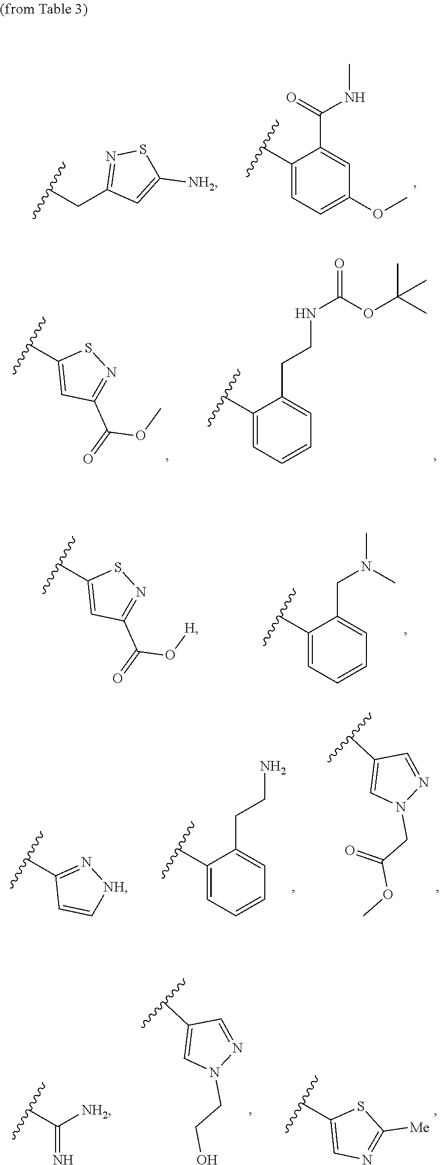

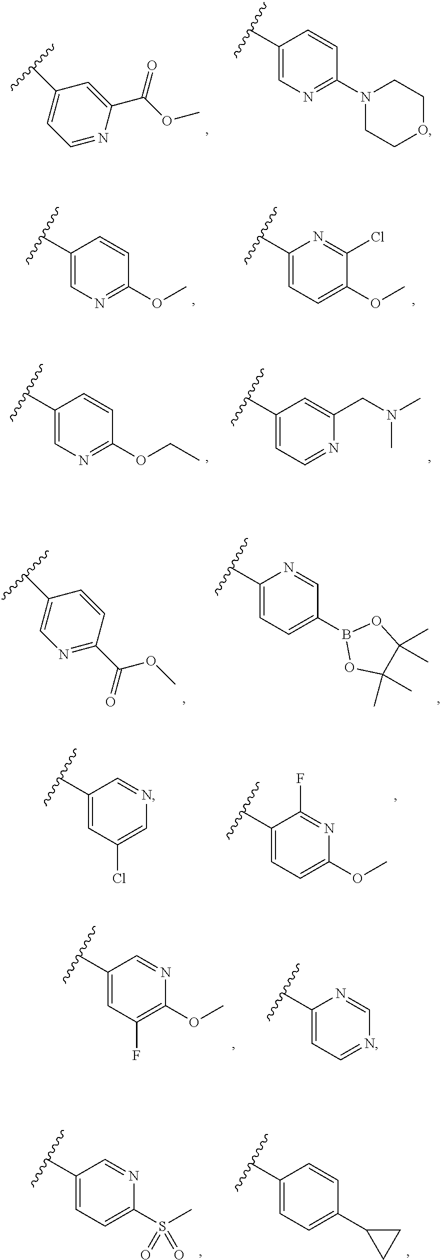

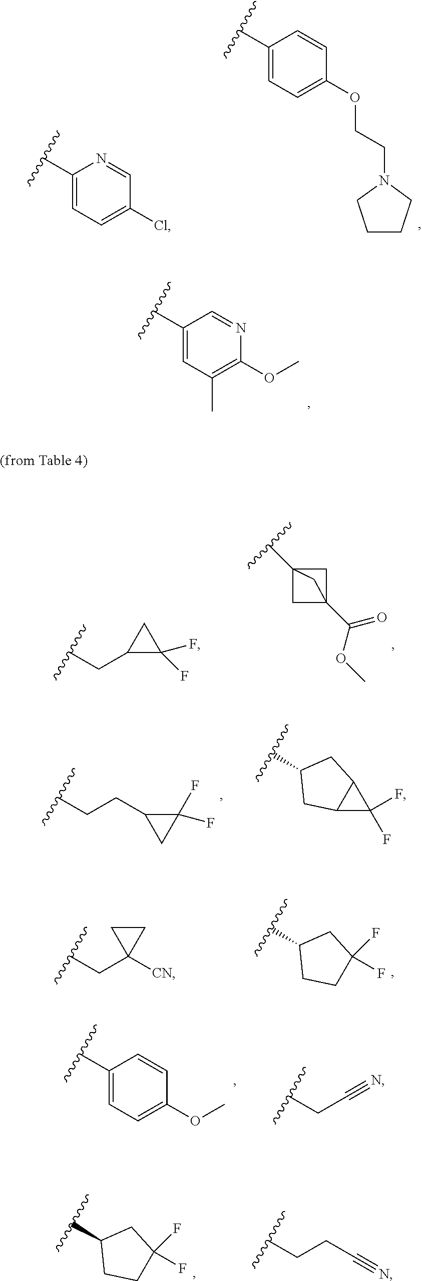

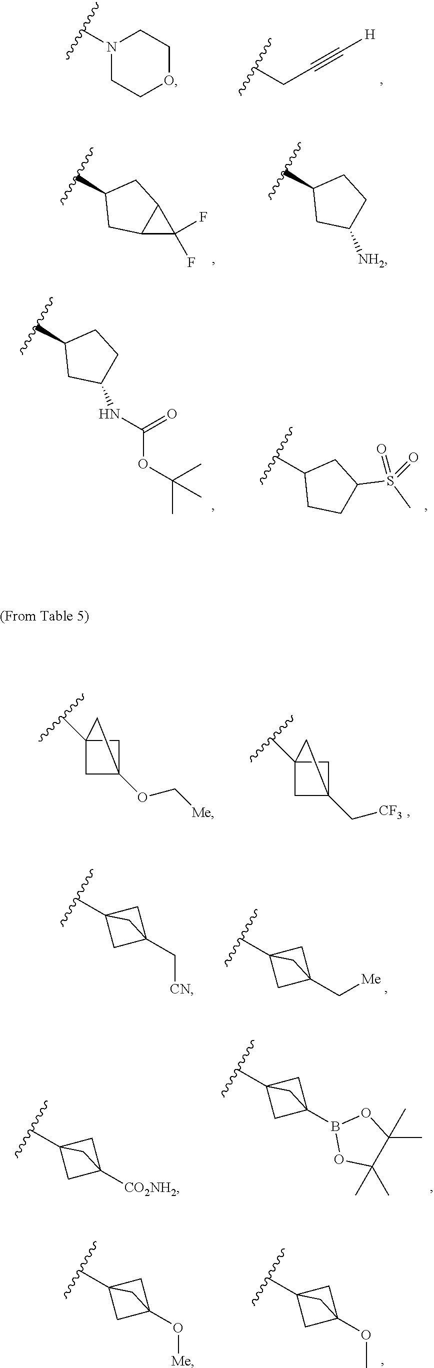

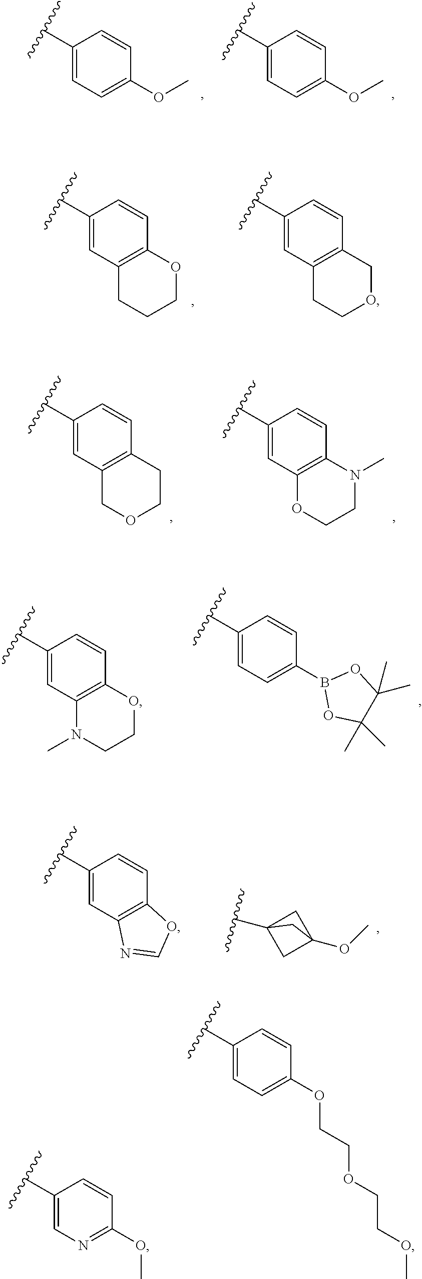

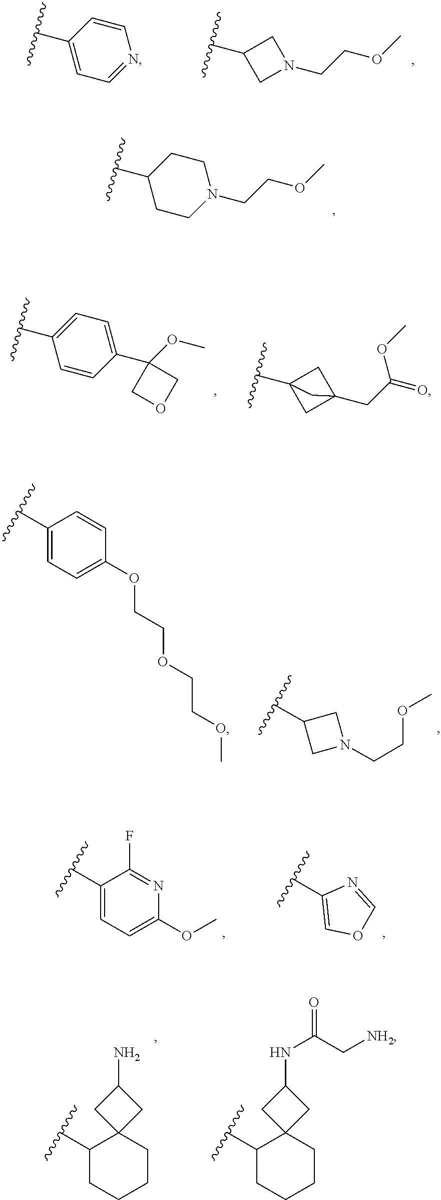

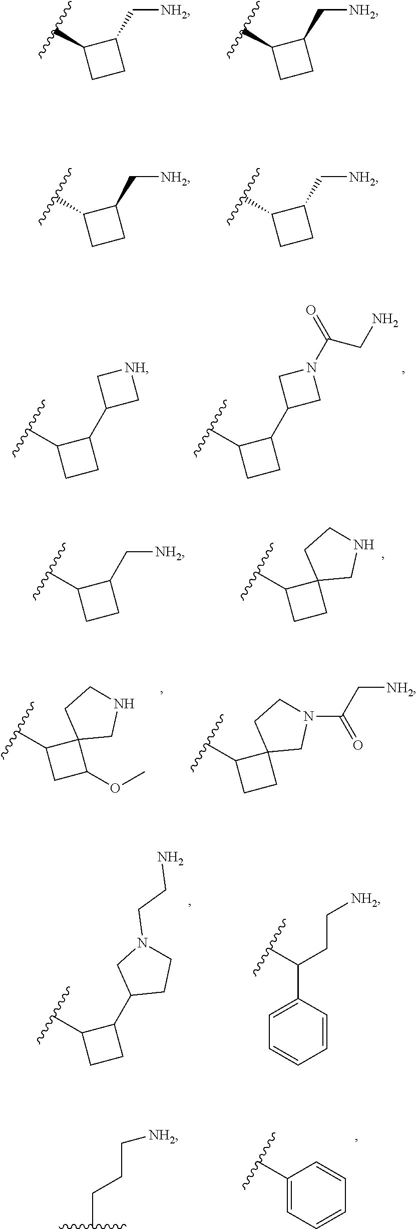

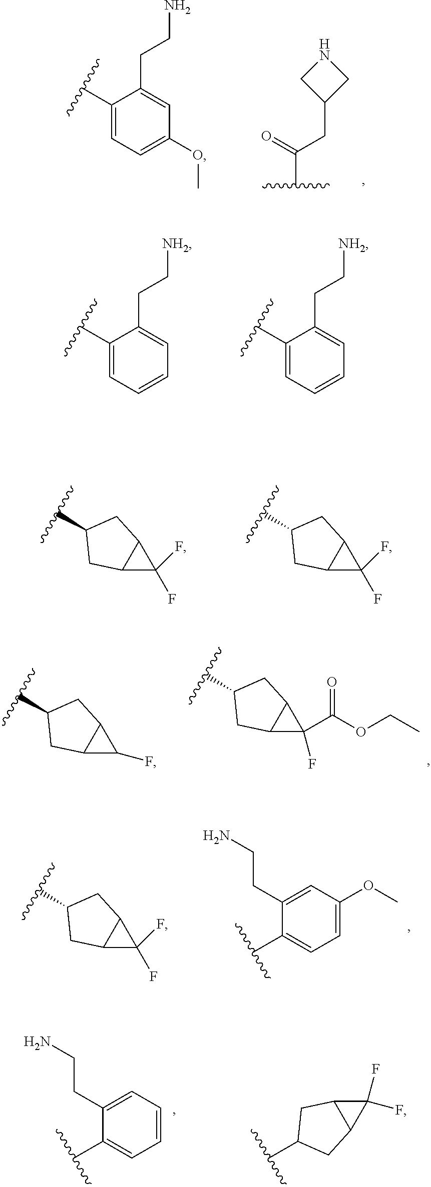

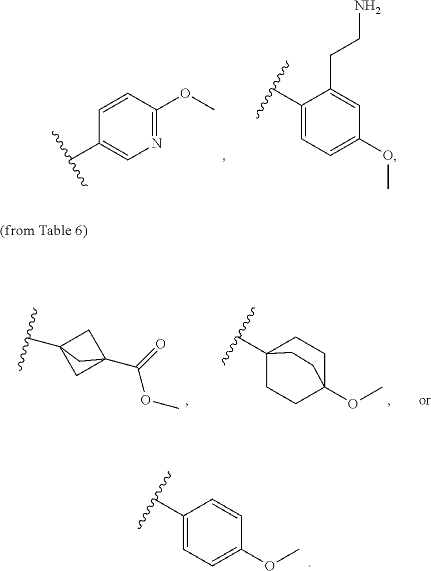

In some embodiments, J² is

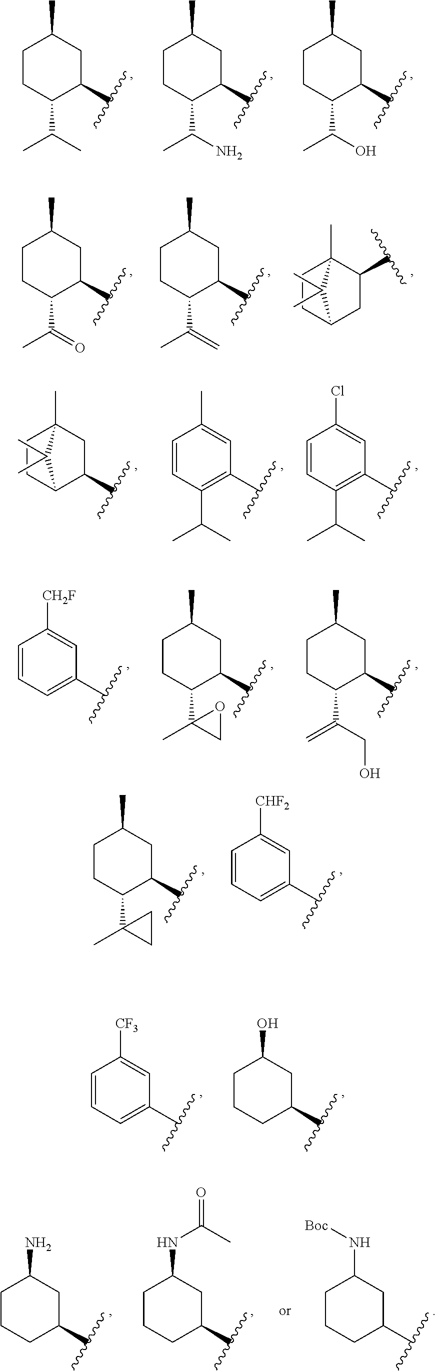

In some embodiments, J⁴ is H,

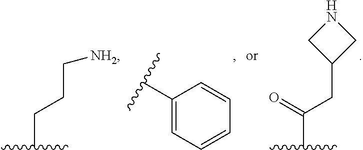

In some embodiments, J¹ and J⁴, together with the nitrogen to which they are attached, form

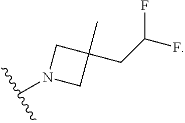 [SMILES: *N1CC(C)(CC(F)F)C1]

In some embodiments, the compounds provided herein are selected from those of Table 1, or a pharmaceutically acceptable salt thereof.

TABLE 1

| Compound | |
|---|---|
| Number | Compound Structure |
| 1 | 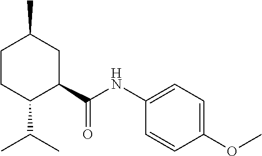 [SMILES: COc1ccc(NC(=O)[C@@H]2C[C@H](C)CC[C@H]2C(C)C)cc1] |
| 2 | 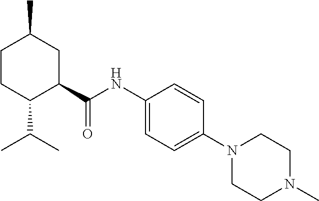 [SMILES: CC(C)[C@@H]1CC[C@@H](C)C[C@H]1C(=O)Nc1ccc(N2CCN(C)CC2)cc1] |
| 3 | 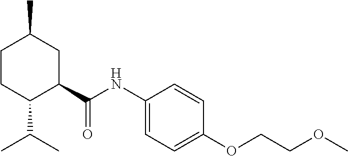 [SMILES: COCCOc1ccc(NC(=O)[C@@H]2C[C@H](C)CC[C@H]2C(C)C)cc1] |
| 4 | 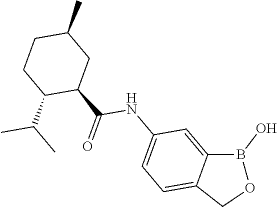 [SMILES: CC(C)[C@@H]1CC[C@@H](C)C[C@H]1C(=O)Nc1ccc2c(c1)B(O)OC2] |
| 5 | 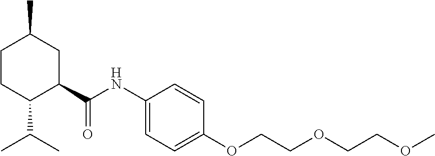 [SMILES: COCCOCCOc1ccc(NC(=O)[C@@H]2C[C@H](C)CC[C@H]2C(C)C)cc1] |
| 6 | 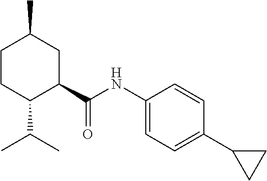 [SMILES: CC(C)[C@@H]1CC[C@@H](C)C[C@H]1C(=O)Nc1ccc(C2CC2)cc1] |
| 7 | 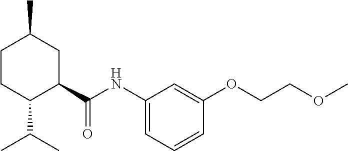 [SMILES: COCCOc1cccc(NC(=O)[C@@H]2C[C@H](C)CC[C@H]2C(C)C)c1] |
| 8 | 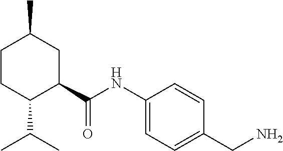 [SMILES: CC(C)[C@@H]1CC[C@@H](C)C[C@H]1C(=O)Nc1ccc(CN)cc1] |
| 9 | 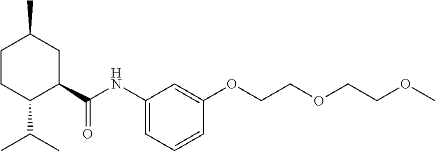 [SMILES: COCCOCCOc1cccc(NC(=O)[C@@H]2C[C@H](C)CC[C@H]2C(C)C)c1] |
| 10 | 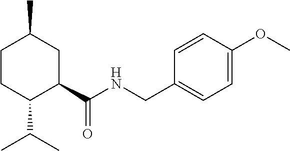 [SMILES: COc1ccc(CNC(=O)[C@@H]2C[C@H](C)CC[C@H]2C(C)C)cc1] |
| 11 | 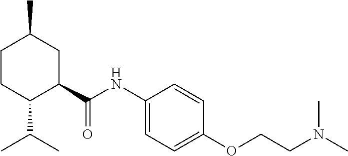 [SMILES: CC(C)[C@@H]1CC[C@@H](C)C[C@H]1C(=O)Nc1ccc(OCCN(C)C)cc1] |
| 12 | 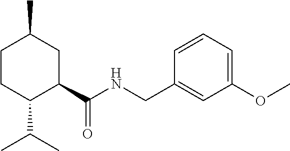 [SMILES: COc1cccc(CNC(=O)[C@@H]2C[C@H](C)CC[C@H]2C(C)C)c1] |
| 13 | 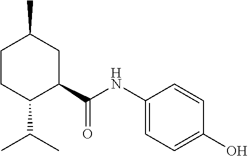 [SMILES: CC(C)[C@@H]1CC[C@@H](C)C[C@H]1C(=O)Nc1ccc(O)cc1] |
| 14 | 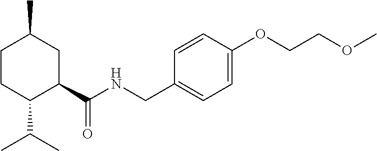 [SMILES: COCCOc1ccc(CNC(=O)[C@@H]2C[C@H](C)CC[C@H]2C(C)C)cc1] |
| 15 | 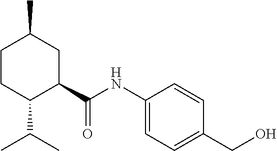 [SMILES: CC(C)[C@@H]1CC[C@@H](C)C[C@H]1C(=O)Nc1ccc(CO)cc1] |
| 16 | 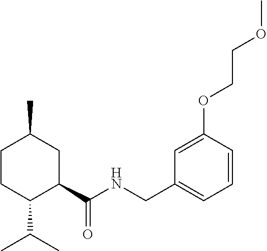 [SMILES: COCCOc1cccc(CNC(=O)[C@@H]2C[C@H](C)CC[C@H]2C(C)C)c1] |
| 17 | 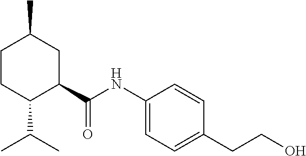 [SMILES: CC(C)[C@@H]1CC[C@@H](C)C[C@H]1C(=O)Nc1ccc(CCO)cc1] |
| 18 | 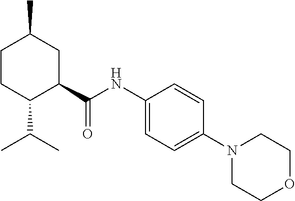 [SMILES: CC(C)[C@@H]1CC[C@@H](C)C[C@H]1C(=O)Nc1ccc(N2CCOCC2)cc1] |
| 19 | 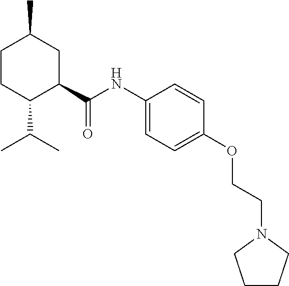 [SMILES: CC(C)[C@@H]1CC[C@@H](C)C[C@H]1C(=O)Nc1ccc(OCCN2CCCC2)cc1] |
| 20 | 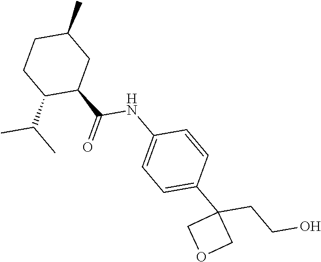 [SMILES: CC(C)[C@@H]1CC[C@@H](C)C[C@H]1C(=O)Nc1ccc(C2(CCO)COC2)cc1] |
| 21 | 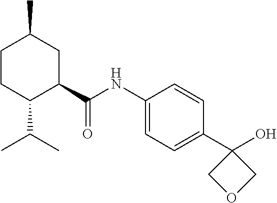 [SMILES: CC(C)[C@@H]1CC[C@@H](C)C[C@H]1C(=O)Nc1ccc(C2(O)COC2)cc1] |
| 22 | 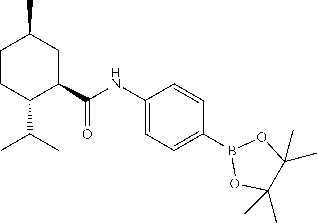 [SMILES: CC(C)[C@@H]1CC[C@@H](C)C[C@H]1C(=O)Nc1ccc(B2OC(C)(C)C(C)(C)O2)cc1] |
| 23 | 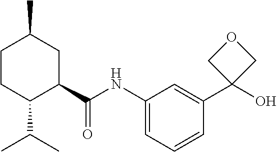 [SMILES: CC(C)[C@@H]1CC[C@@H](C)C[C@H]1C(=O)Nc1cccc(C2(O)COC2)c1] |
| 24 | 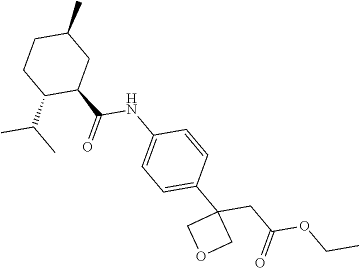 [SMILES: CCOC(=O)CC1(c2ccc(NC(=O)[C@@H]3C[C@H](C)CC[C@H]3C(C)C)cc2)COC1] |

In some embodiments, the compounds provided herein are selected from those of Table 2, or a pharmaceutically acceptable salt thereof.

TABLE 2

| Compound | Compound |
|---|---|
| Number | Structure |
| 25 | 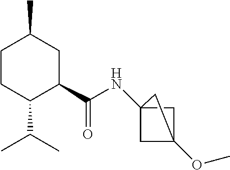 [SMILES: COC12CC(NC(=O)[C@@H]3C[C@H](C)CC[C@H]3C(C)C)(C1)C2] |
| 26 | 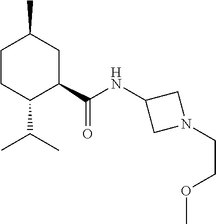 [SMILES: COCCN1CC(NC(=O)[C@@H]2C[C@H](C)CC[C@H]2C(C)C)C1] |
| 27 | 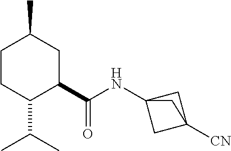 [SMILES: CC(C)[C@@H]1CC[C@@H](C)C[C@H]1C(=O)NC12CC(C#N)(C1)C2] |
| 28 | 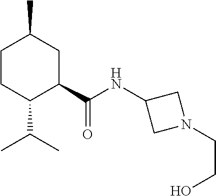 [SMILES: CC(C)[C@@H]1CC[C@@H](C)C[C@H]1C(=O)NC1CN(CCO)C1] |
| 29 | 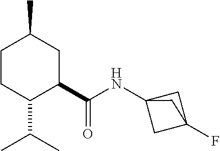 [SMILES: CC(C)[C@@H]1CC[C@@H](C)C[C@H]1C(=O)NC12CC(F)(C1)C2] |
| 30 | 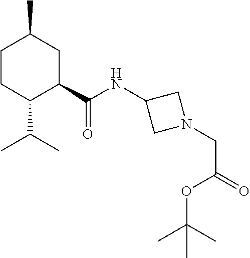 [SMILES: CC(C)[C@@H]1CC[C@@H](C)C[C@H]1C(=O)NC1CN(CC(=O)OC(C)(C)C)C1] |
| 31 | 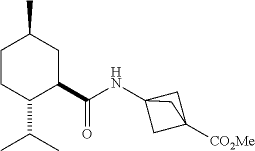 [SMILES: COC(=O)C12CC(NC(=O)[C@@H]3C[C@H](C)CC[C@H]3C(C)C)(C1)C2] |
| 32 | 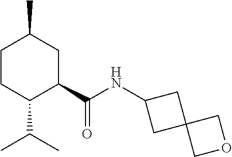 [SMILES: CC(C)[C@@H]1CC[C@@H](C)C[C@H]1C(=O)NC1CC2(COC2)C1] |
| 33 | 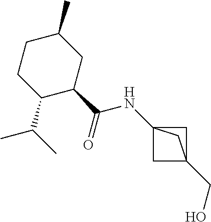 [SMILES: CC(C)[C@@H]1CC[C@@H](C)C[C@H]1C(=O)NC12CC(CO)(C1)C2] |
| 34 | 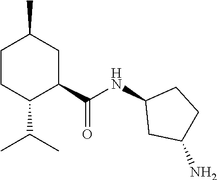 [SMILES: CC(C)[C@@H]1CC[C@@H](C)C[C@H]1C(=O)N[C@H]1CC[C@H](N)C1] |
| 35 | 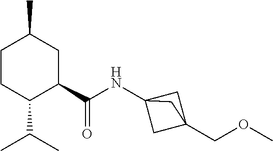 [SMILES: COCC12CC(NC(=O)[C@@H]3C[C@H](C)CC[C@H]3C(C)C)(C1)C2] |
| 36 | 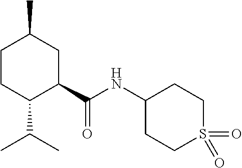 [SMILES: CC(C)[C@@H]1CC[C@@H](C)C[C@H]1C(=O)NC1CCS(=O)(=O)CC1] |
| 37 | 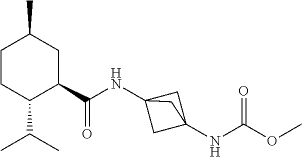 [SMILES: COC(=O)NC12CC(NC(=O)[C@@H]3C[C@H](C)CC[C@H]3C(C)C)(C1)C2] |
| 38 | 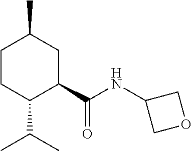 [SMILES: CC(C)[C@@H]1CC[C@@H](C)C[C@H]1C(=O)NC1COC1] |
| 39 | 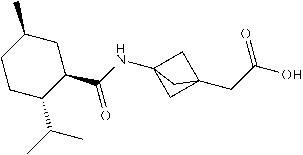 [SMILES: CC(C)[C@@H]1CC[C@@H](C)C[C@H]1C(=O)NC12CC(CC(=O)O)(C1)C2] |
| 40 | 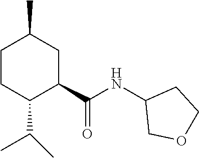 [SMILES: CC(C)[C@@H]1CC[C@@H](C)C[C@H]1C(=O)NC1CCOC1] |
| 41 | 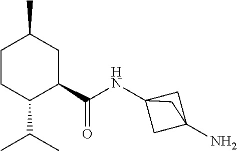 [SMILES: CC(C)[C@@H]1CC[C@@H](C)C[C@H]1C(=O)NC12CC(N)(C1)C2] |
| 42 | 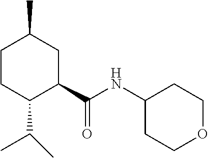 [SMILES: CC(C)[C@@H]1CC[C@@H](C)C[C@H]1C(=O)NC1CCOCC1] |
| 43 | 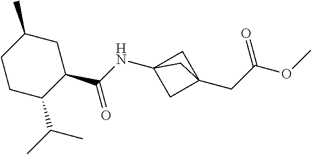 [SMILES: COC(=O)CC12CC(NC(=O)[C@@H]3C[C@H](C)CC[C@H]3C(C)C)(C1)C2] |
| 44 | 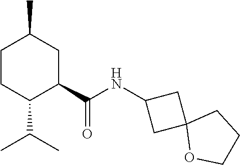 [SMILES: CC(C)[C@@H]1CC[C@@H](C)C[C@H]1C(=O)NC1CC2(CCCO2)C1] |
| 45 | 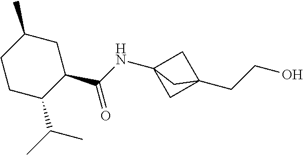 [SMILES: CC(C)[C@@H]1CC[C@@H](C)C[C@H]1C(=O)NC12CC(CCO)(C1)C2] |
| 46 | 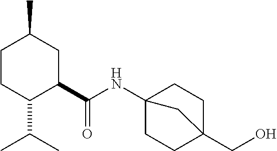 [SMILES: CC(C)[C@@H]1CC[C@@H](C)C[C@H]1C(=O)NC12CCC(CO)(CC1)C2] |
| 47 | 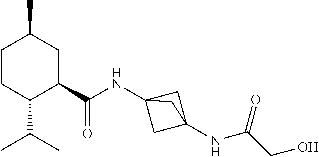 [SMILES: CC(C)[C@@H]1CC[C@@H](C)C[C@H]1C(=O)NC12CC(NC(=O)CO)(C1)C2] |
| 48 | 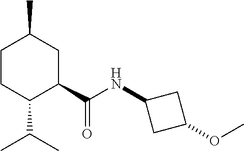 |
| 49 | 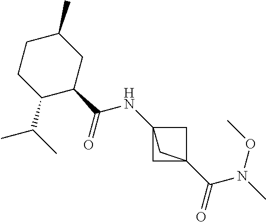 [SMILES: CON(C)C(=O)C12CC(NC(=O)[C@@H]3C[C@H](C)CC[C@H]3C(C)C)(C1)C2] |
| 50 | 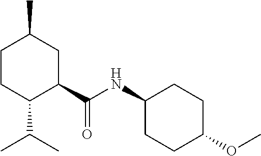 [SMILES: CO[C@H]1CC[C@H](NC(=O)[C@@H]2C[C@H](C)CC[C@H]2C(C)C)CC1] |
| 51 | 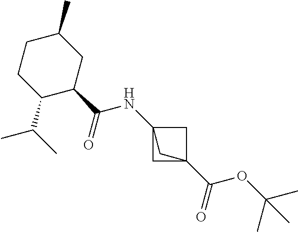 [SMILES: CC(C)[C@@H]1CC[C@@H](C)C[C@H]1C(=O)NC12CC(C(=O)OC(C)(C)C)(C1)C2] |
| 52 | 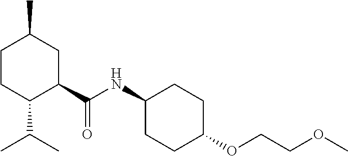 [SMILES: COCCO[C@H]1CC[C@H](NC(=O)[C@@H]2C[C@H](C)CC[C@H]2C(C)C)CC1] |
| 53 | 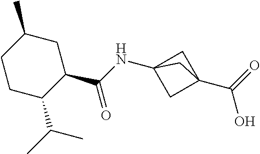 [SMILES: CC(C)[C@@H]1CC[C@@H](C)C[C@H]1C(=O)NC12CC(C(=O)O)(C1)C2] |
| 54 | 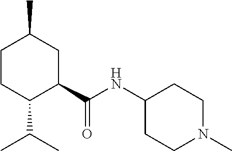 [SMILES: CC(C)[C@@H]1CC[C@@H](C)C[C@H]1C(=O)NC1CCN(C)CC1] |
| 55 | 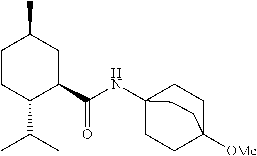 [SMILES: COC12CCC(NC(=O)[C@@H]3C[C@H](C)CC[C@H]3C(C)C)(CC1)CC2] |
| 56 | 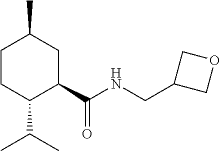 [SMILES: CC(C)[C@@H]1CC[C@@H](C)C[C@H]1C(=O)NCC1COC1] |
| 57 | 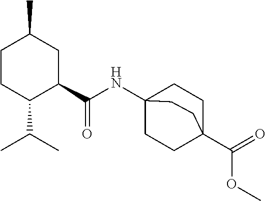 [SMILES: COC(=O)C12CCC(NC(=O)[C@@H]3C[C@H](C)CC[C@H]3C(C)C)(CC1)CC2] |
| 58 | 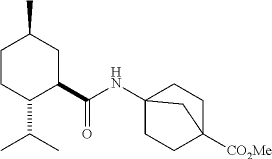 [SMILES: COC(=O)C12CCC(NC(=O)[C@@H]3C[C@H](C)CC[C@H]3C(C)C)(CC1)C2] |
| 59 | 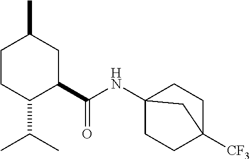 [SMILES: CC(C)[C@@H]1CC[C@@H](C)C[C@H]1C(=O)NC12CCC(C(F)(F)F)(CC1)C2] |

In some embodiments, the compounds provided herein are selected from those of Table 3, or a pharmaceutically acceptable salt thereof.

TABLE 3

| Compound | Compound |
|---|---|
| Number | Structure |
| 60 | 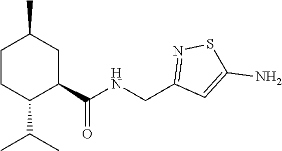 [SMILES: CC(C)[C@@H]1CC[C@@H](C)C[C@H]1C(=O)NCc1cc(N)sn1] |
| 61 | 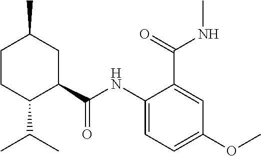 [SMILES: CNC(=O)c1cc(OC)ccc1NC(=O)[C@@H]1C[C@H](C)CC[C@H]1C(C)C] |
| 62 | 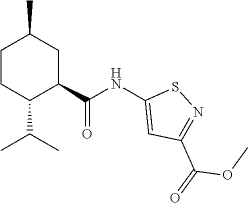 [SMILES: COC(=O)c1cc(NC(=O)[C@@H]2C[C@H](C)CC[C@H]2C(C)C)sn1] |
| 63 | 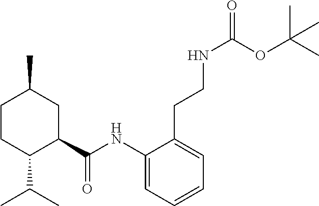 [SMILES: CC(C)[C@@H]1CC[C@@H](C)C[C@H]1C(=O)Nc1ccccc1CCNC(=O)OC(C)(C)C] |
| 64 | 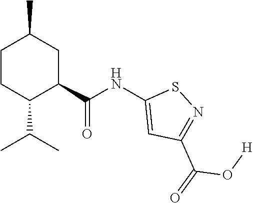 [SMILES: CC(C)[C@@H]1CC[C@@H](C)C[C@H]1C(=O)Nc1cc(C(=O)O)ns1] |
| 65 | 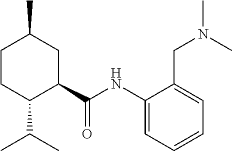 [SMILES: CC(C)[C@@H]1CC[C@@H](C)C[C@H]1C(=O)Nc1ccccc1CN(C)C] |
| 66 | 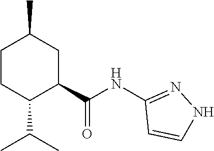 [SMILES: CC(C)[C@@H]1CC[C@@H](C)C[C@H]1C(=O)Nc1cc[nH]n1] |
| 67 | 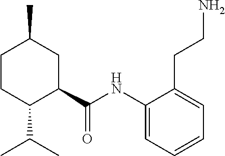 [SMILES: CC(C)[C@@H]1CC[C@@H](C)C[C@H]1C(=O)Nc1ccccc1CCN] |
| 68 | 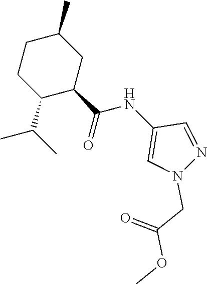 [SMILES: COC(=O)Cn1cc(NC(=O)[C@@H]2C[C@H](C)CC[C@H]2C(C)C)cn1] |
| 69 | 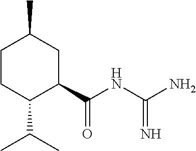 [SMILES: CC(C)[C@@H]1CC[C@@H](C)C[C@H]1C(=O)NC(=N)N] |
| 70 | 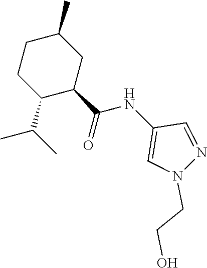 [SMILES: CC(C)[C@@H]1CC[C@@H](C)C[C@H]1C(=O)Nc1cnn(CCO)c1] |
| 71 | 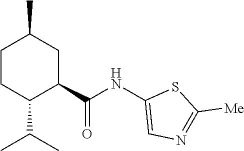 [SMILES: Cc1ncc(NC(=O)[C@@H]2C[C@H](C)CC[C@H]2C(C)C)s1] |
| 72 | 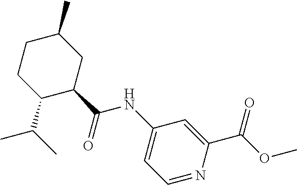 [SMILES: COC(=O)c1cc(NC(=O)[C@@H]2C[C@H](C)CC[C@H]2C(C)C)ccn1] |
| 73 | 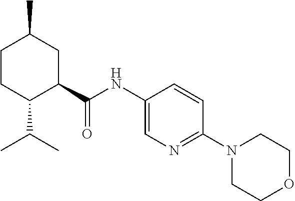 [SMILES: CC(C)[C@@H]1CC[C@@H](C)C[C@H]1C(=O)Nc1ccc(N2CCOCC2)nc1] |
| 74 | 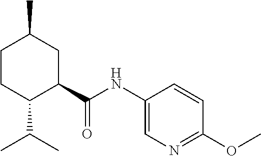 [SMILES: COc1ccc(NC(=O)[C@@H]2C[C@H](C)CC[C@H]2C(C)C)cn1] |
| 75 | 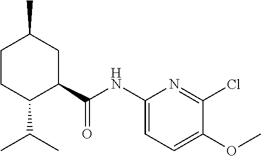 [SMILES: COc1ccc(NC(=O)[C@@H]2C[C@H](C)CC[C@H]2C(C)C)nc1Cl] |
| 76 | 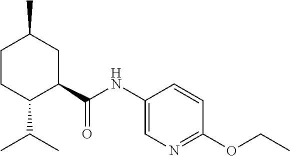 [SMILES: CCOc1ccc(NC(=O)[C@@H]2C[C@H](C)CC[C@H]2C(C)C)cn1] |
| 77 | 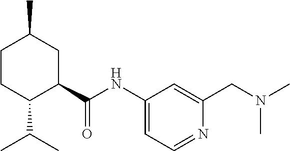 [SMILES: CC(C)[C@@H]1CC[C@@H](C)C[C@H]1C(=O)Nc1ccnc(CN(C)C)c1] |
| 78 | 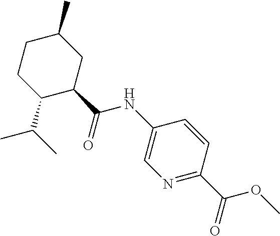 [SMILES: COC(=O)c1ccc(NC(=O)[C@@H]2C[C@H](C)CC[C@H]2C(C)C)cn1] |
| 79 | 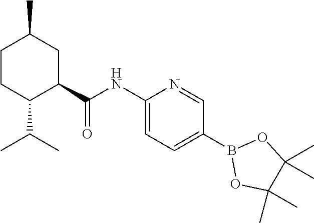 [SMILES: CC(C)[C@@H]1CC[C@@H](C)C[C@H]1C(=O)Nc1ccc(B2OC(C)(C)C(C)(C)O2)cn1] |
| 80 | 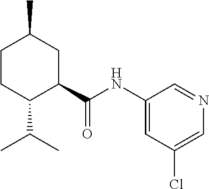 [SMILES: CC(C)[C@@H]1CC[C@@H](C)C[C@H]1C(=O)Nc1cncc(Cl)c1] |
| 81 | 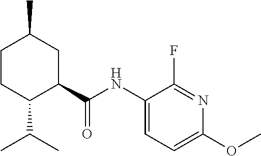 [SMILES: COc1ccc(NC(=O)[C@@H]2C[C@H](C)CC[C@H]2C(C)C)c(F)n1] |
| 82 | 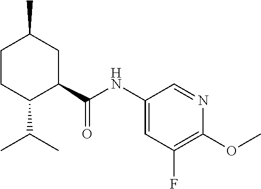 [SMILES: COc1ncc(NC(=O)[C@@H]2C[C@H](C)CC[C@H]2C(C)C)cc1F] |
| 83 | 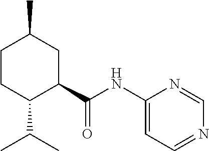 [SMILES: CC(C)[C@@H]1CC[C@@H](C)C[C@H]1C(=O)Nc1ccncn1] |
| 84 | 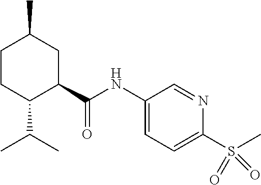 [SMILES: CC(C)[C@@H]1CC[C@@H](C)C[C@H]1C(=O)Nc1ccc(S(C)(=O)=O)nc1] |
| 85 | 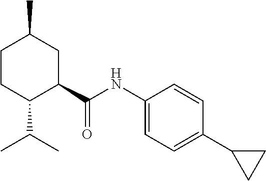 [SMILES: CC(C)[C@@H]1CC[C@@H](C)C[C@H]1C(=O)Nc1ccc(C2CC2)cc1] |
| 86 | 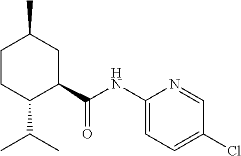 [SMILES: CC(C)[C@@H]1CC[C@@H](C)C[C@H]1C(=O)Nc1ccc(Cl)cn1] |
| 87 | 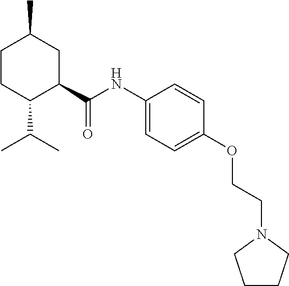 [SMILES: CC(C)[C@@H]1CC[C@@H](C)C[C@H]1C(=O)Nc1ccc(OCCN2CCCC2)cc1] |
| 88 | 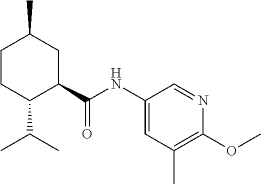 [SMILES: COc1ncc(NC(=O)[C@@H]2C[C@H](C)CC[C@H]2C(C)C)cc1C] |

In some embodiments, the compounds provided herein are selected from those of Table 4, or a pharmaceutically acceptable salt thereof.

TABLE 4

| Compound | Compound |
|---|---|
| Number | Structure |
| 89 | 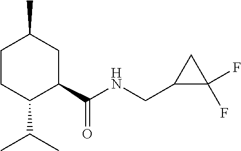 [SMILES: CC(C)[C@@H]1CC[C@@H](C)C[C@H]1C(=O)NCC1CC1(F)F] |
| 90 | 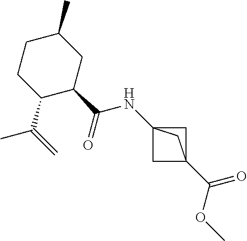 [SMILES: C=C(C)[C@@H]1CC[C@@H](C)C[C@H]1C(=O)NC12CC(C(=O)OC)(C1)C2] |
| 91 | 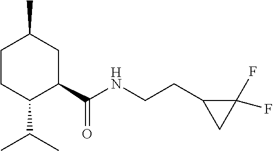 [SMILES: CC(C)[C@@H]1CC[C@@H](C)C[C@H]1C(=O)NCCC1CC1(F)F] |
| 92 | 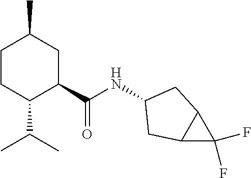 |
| 93 | 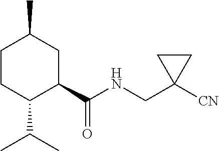 [SMILES: CC(C)[C@@H]1CC[C@@H](C)C[C@H]1C(=O)NCC1(C#N)CC1] |
| 94 | 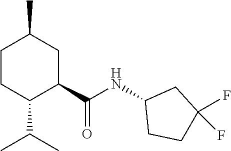 [SMILES: CC(C)[C@@H]1CC[C@@H](C)C[C@H]1C(=O)N[C@H]1CCC(F)(F)C1] |
| 95 | 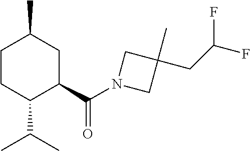 [SMILES: CC(C)[C@@H]1CC[C@@H](C)C[C@H]1C(=O)N1CC(C)(CC(F)F)C1] |
| 96 | 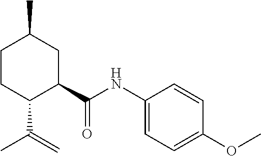 [SMILES: C=C(C)[C@@H]1CC[C@@H](C)C[C@H]1C(=O)Nc1ccc(OC)cc1] |
| 97 | 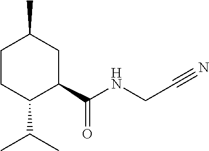 [SMILES: CC(C)[C@@H]1CC[C@@H](C)C[C@H]1C(=O)NCC#N] |
| 98 | 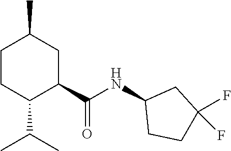 [SMILES: CC(C)[C@@H]1CC[C@@H](C)C[C@H]1C(=O)N[C@@H]1CCC(F)(F)C1] |
| 99 | 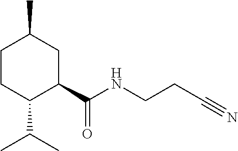 [SMILES: CC(C)[C@@H]1CC[C@@H](C)C[C@H]1C(=O)NCCC#N] |
| 100 | 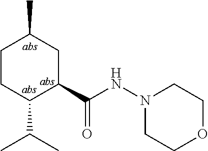 [SMILES: CC(C)[C@@H]1CC[C@@H](C)C[C@H]1C(=O)NN1CCOCC1] |
| 101 | 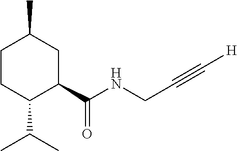 [SMILES: C#CCNC(=O)[C@@H]1C[C@H](C)CC[C@H]1C(C)C] |
| 102 | 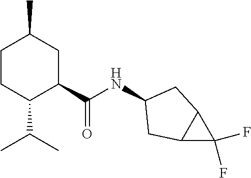 |
| 103 | 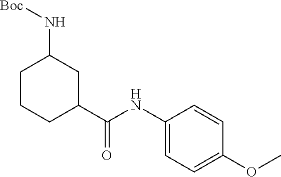 [SMILES: COc1ccc(NC(=O)C2CCCC(NC(=O)OC(C)(C)C)C2)cc1] |
| 104 | 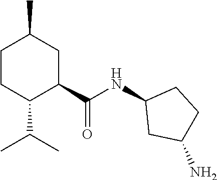 [SMILES: CC(C)[C@@H]1CC[C@@H](C)C[C@H]1C(=O)N[C@H]1CC[C@H](N)C1] |
| 105 | 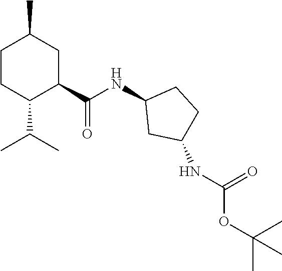 [SMILES: CC(C)[C@@H]1CC[C@@H](C)C[C@H]1C(=O)N[C@H]1CC[C@H](NC(=O)OC(C)(C)C)C1] |
| 106 | 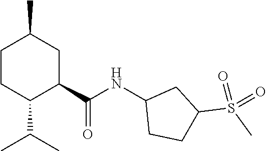 [SMILES: CC(C)[C@@H]1CC[C@@H](C)C[C@H]1C(=O)NC1CCC(S(C)(=O)=O)C1] |
| 107 | 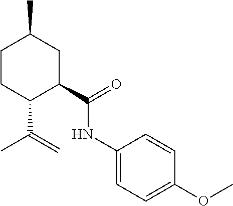 [SMILES: C=C(C)[C@@H]1CC[C@@H](C)C[C@H]1C(=O)Nc1ccc(OC)cc1] |
| 108 | 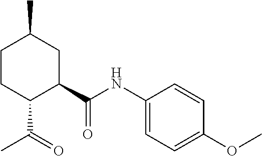 [SMILES: COc1ccc(NC(=O)[C@@H]2C[C@H](C)CC[C@H]2C(C)=O)cc1] |
| 109 | 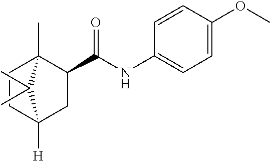 |

In some embodiments, the compounds provided herein are selected from those of Table 5, or a pharmaceutically acceptable salt thereof.

TABLE 5

| Com- | |
|---|---|
| pound | |
| Number | Compound Structure |
| 110 | 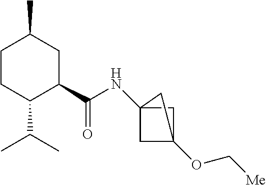 [SMILES: CCOC12CC(NC(=O)[C@@H]3C[C@H](C)CC[C@H]3C(C)C)(C1)C2] |
| 111 | 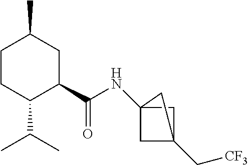 [SMILES: CC(C)[C@@H]1CC[C@@H](C)C[C@H]1C(=O)NC12CC(CC(F)(F)F)(C1)C2] |
| 112 | 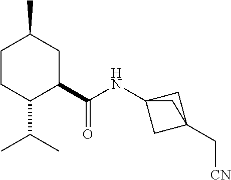 [SMILES: CC(C)[C@@H]1CC[C@@H](C)C[C@H]1C(=O)NC12CC(CC#N)(C1)C2] |
| 113 | 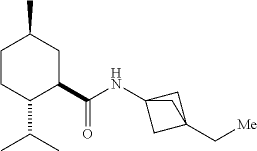 [SMILES: CCC12CC(NC(=O)[C@@H]3C[C@H](C)CC[C@H]3C(C)C)(C1)C2] |
| 114 | 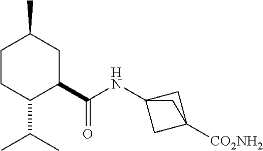 |
| 115 | 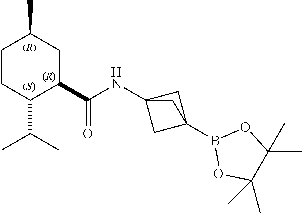 |
| 116 | 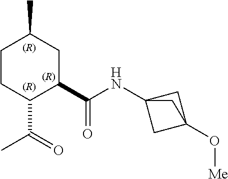 |
| 117 | 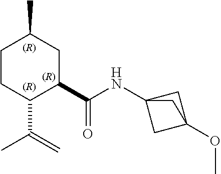 |
| 118 | 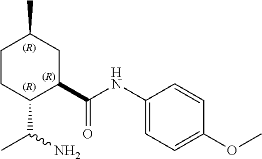 |
| 119 | 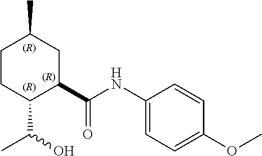 |
| 120 | 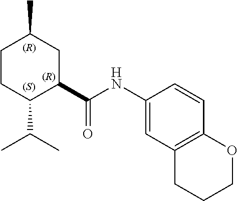 |
| 121 | 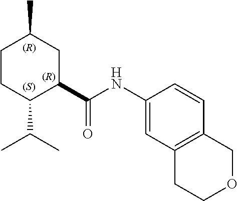 |
| 122 | 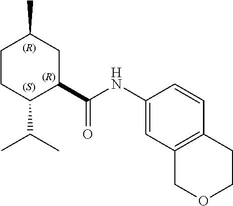 |
| 123 | 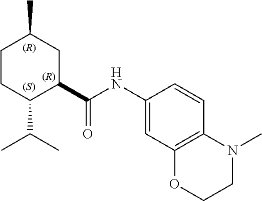 |
| 124 | 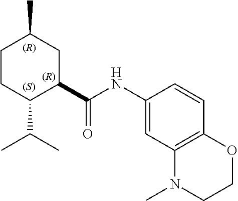 |
| 125 | 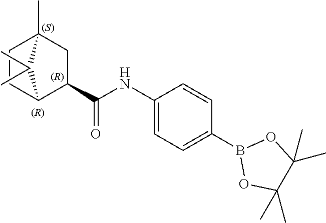 |
| 126 | 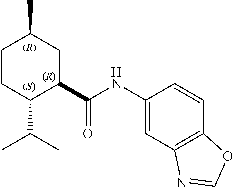 |
| 127 | 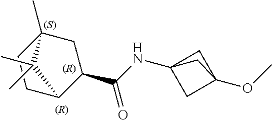 |
| 128 | 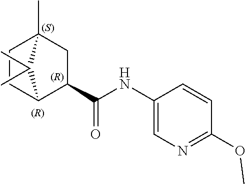 |
| 129 | 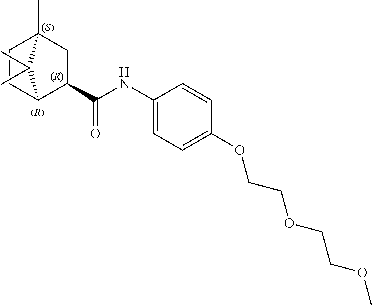 |
| 130 | 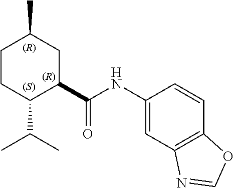 |
| 131 | 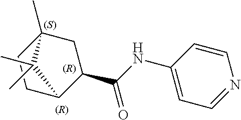 |
| 132 | 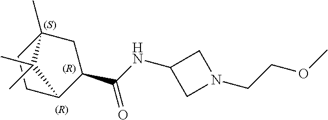 |
| 133 | 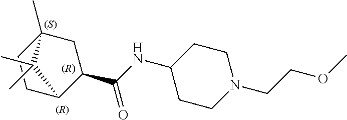 |
| 134 | 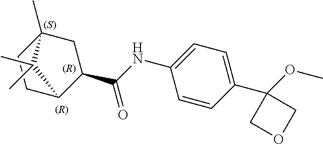 |
| 135 | 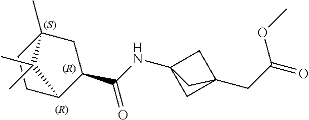 |
| 136 | 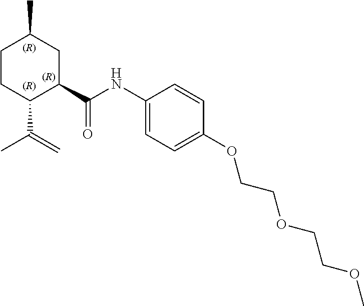 |
| 137 | 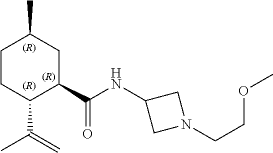 |
| 138 | 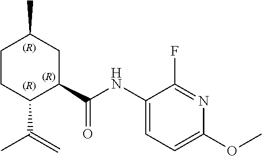 |
| 139 | 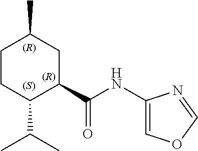 |
| 140 | 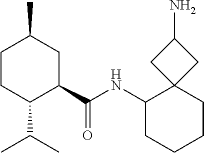 [SMILES: CC(C)[C@@H]1CC[C@@H](C)C[C@H]1C(=O)NC1CCCCC12CC(N)C2] |
| 141 | 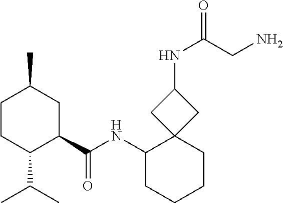 [SMILES: CC(C)[C@@H]1CC[C@@H](C)C[C@H]1C(=O)NC1CCCCC12CC(NC(=O)CN)C2] |
| 142 | 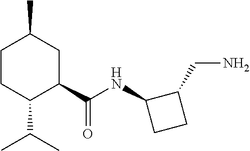 [SMILES: CC(C)[C@@H]1CC[C@@H](C)C[C@H]1C(=O)N[C@@H]1CC[C@H]1CN] |
| 143 | 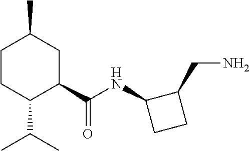 [SMILES: CC(C)[C@@H]1CC[C@@H](C)C[C@H]1C(=O)N[C@@H]1CC[C@@H]1CN] |
| 144 | 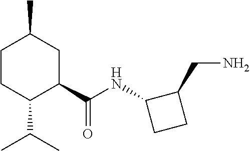 [SMILES: CC(C)[C@@H]1CC[C@@H](C)C[C@H]1C(=O)N[C@H]1CC[C@@H]1CN] |
| 145 | 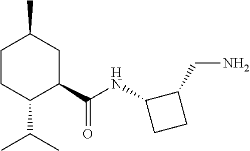 [SMILES: CC(C)[C@@H]1CC[C@@H](C)C[C@H]1C(=O)N[C@H]1CC[C@H]1CN] |
| 146 | 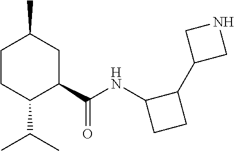 [SMILES: CC(C)[C@@H]1CC[C@@H](C)C[C@H]1C(=O)NC1CCC1C1CNC1] |
| 147 | 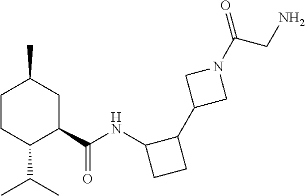 [SMILES: CC(C)[C@@H]1CC[C@@H](C)C[C@H]1C(=O)NC1CCC1C1CN(C(=O)CN)C1] |
| 148 | 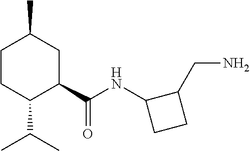 [SMILES: CC(C)[C@@H]1CC[C@@H](C)C[C@H]1C(=O)NC1CCC1CN] |
| 149 | 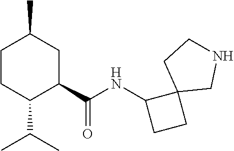 [SMILES: CC(C)[C@@H]1CC[C@@H](C)C[C@H]1C(=O)NC1CCC12CCNC2] |
| 150 | 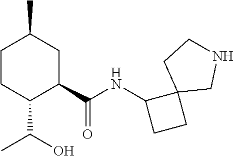 [SMILES: CC(O)[C@@H]1CC[C@@H](C)C[C@H]1C(=O)NC1CCC12CCNC2] |
| 151 | 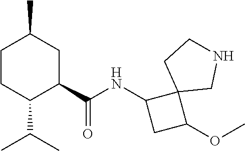 [SMILES: COC1CC(NC(=O)[C@@H]2C[C@H](C)CC[C@H]2C(C)C)C12CCNC2] |
| 152 | 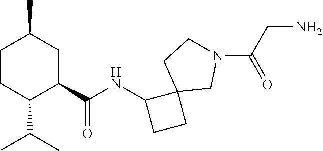 [SMILES: CC(C)[C@@H]1CC[C@@H](C)C[C@H]1C(=O)NC1CCC12CCN(C(=O)CN)C2] |
| 153 | 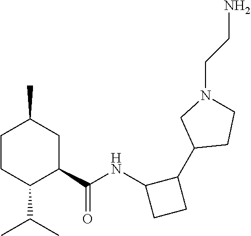 [SMILES: CC(C)[C@@H]1CC[C@@H](C)C[C@H]1C(=O)NC1CCC1C1CCN(CCN)C1] |
| 154 | 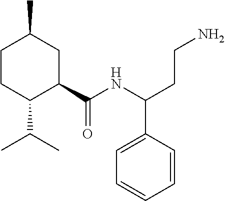 [SMILES: CC(C)[C@@H]1CC[C@@H](C)C[C@H]1C(=O)NC(CCN)c1ccccc1] |
| 155 | 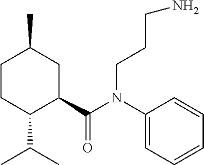 [SMILES: CC(C)[C@@H]1CC[C@@H](C)C[C@H]1C(=O)N(CCCN)c1ccccc1] |
| 156 | 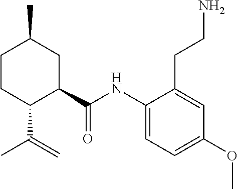 [SMILES: C=C(C)[C@@H]1CC[C@@H](C)C[C@H]1C(=O)Nc1ccc(OC)cc1CCN] |
| 157 | 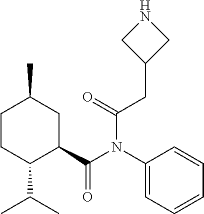 [SMILES: CC(C)[C@@H]1CC[C@@H](C)C[C@H]1C(=O)N(C(=O)CC1CNC1)c1ccccc1] |
| 158 | 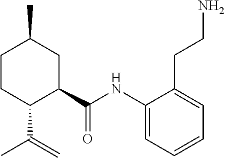 [SMILES: C=C(C)[C@@H]1CC[C@@H](C)C[C@H]1C(=O)Nc1ccccc1CCN] |
| 159 | 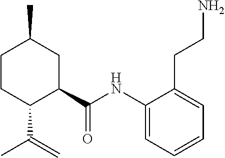 [SMILES: C=C(C)[C@@H]1CC[C@@H](C)C[C@H]1C(=O)Nc1ccccc1CCN] |
| 160 | 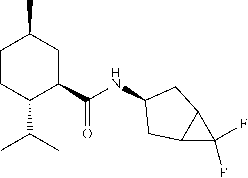 |
| 161 | 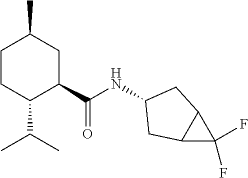 |
| 162 | 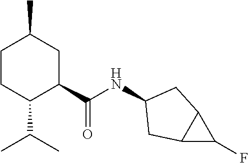 |
| 163 | 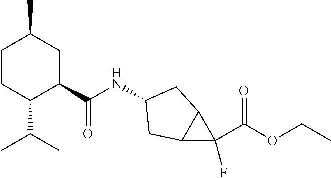 |
| 164 | 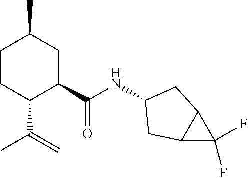 |
| 165 | 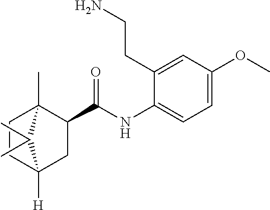 |
| 166 | 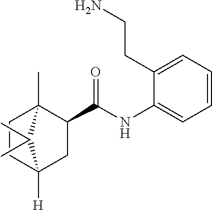 |
| 167 | 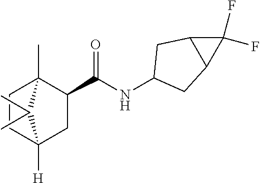 |
| 168 | 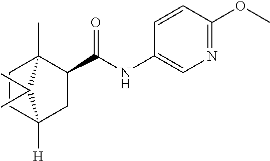 |
| 169 | 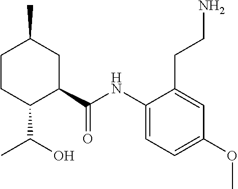 [SMILES: COc1ccc(NC(=O)[C@@H]2C[C@H](C)CC[C@H]2C(C)O)c(CCN)c1] |

In some embodiments, the compounds provided herein are selected from those of Table 6, or a pharmaceutically acceptable salt thereof.

TABLE 6

| Compound | Compound |
|---|---|
| Number | Structure |
| 170 | 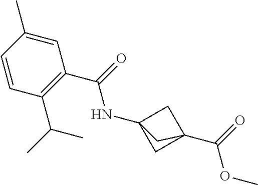 [SMILES: COC(=O)C12CC(NC(=O)c3cc(C)ccc3C(C)C)(C1)C2] |
| 171 | 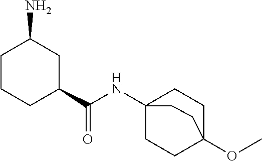 [SMILES: COC12CCC(NC(=O)[C@H]3CCC[C@@H](N)C3)(CC1)CC2] |
| 172 | 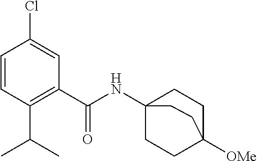 [SMILES: COC12CCC(NC(=O)c3cc(Cl)ccc3C(C)C)(CC1)CC2] |
| 173 | 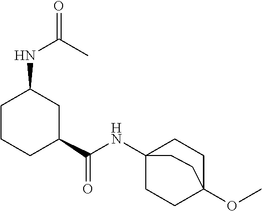 [SMILES: COC12CCC(NC(=O)[C@H]3CCC[C@@H](NC(C)=O)C3)(CC1)CC2] |
| 174 | 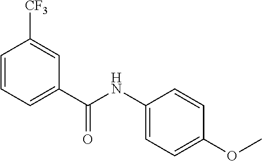 [SMILES: COc1ccc(NC(=O)c2cccc(C(F)(F)F)c2)cc1] |
| 175 | 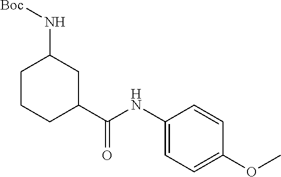 [SMILES: COc1ccc(NC(=O)C2CCCC(NC(=O)OC(C)(C)C)C2)cc1] |
| 176 | 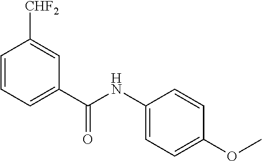 [SMILES: COc1ccc(NC(=O)c2cccc(C(F)F)c2)cc1] |
| 177 | 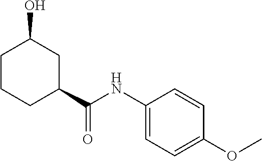 [SMILES: COc1ccc(NC(=O)[C@H]2CCC[C@@H](O)C2)cc1] |
| 178 | 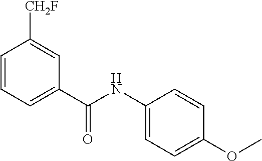 |

The present disclosure also includes isotopically-labeled compounds, which are identical to those recited in the formulae herein, but for the fact that one or more atoms are replaced by an atom having an atomic mass or mass number abundance different from the atomic mass or mass number abundance usually found in nature. Examples of isotopes suitable for inclusion in the compounds of this invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain therapeutic advantages resulting from greater metabolic stability, for example, increased in vivo half-life or reduced dosage requirements and hence may be preferred in some circumstances. The compound may incorporate positron-emitting isotopes for medical imaging and positron-emitting tomography (PET) studies for determining the distribution of receptors. Suitable positron-emitting isotopes that can be incorporated in the compounds herein are ¹¹C, ¹³N, ¹⁵O, and ¹⁸F. Compounds herein that are isotopically-labeled can generally be prepared by conventional techniques known to those skilled in the art or by processes analogous to those described herein using appropriate isotopically-labeled reagents or starting materials in place of non-isotopically-labeled reagents or starting materials. Thus, in some embodiments, provided herein are compounds of the formulae herein, wherein at least one atom is replaced by its corresponding atom having an atomic mass or mass number abundance different from the atomic mass or mass number abundance usually found in nature. For example, the compounds have one or more H (e.g., ¹H) substituted, independently, with ²H or ³H. In some embodiments, one or more C is substituted, independently, with ¹³C or ¹⁴C. In some embodiments, one or more N is substituted with ¹⁵N. In some embodiments, one or more O is substituted, independently, with ¹⁸O or ¹⁷O. In some embodiments, one or more P is substituted, independently, with ³²P. In some embodiments, one or more S is substituted, independently, with ³⁵S. In some embodiments, one or more F is substituted, independently, with ¹⁸F. In some embodiments, one or more Cl is substituted, independently, with ³⁵Cl, ³⁶Cl, or ³⁷Cl.

The disclosed compounds may exist as pharmaceutically acceptable salts. The term “pharmaceutically acceptable salt” refers to salts or zwitterions of the compounds that are suitable for therapeutic use without undue toxicity, irritation, or allergic response, commensurate with a reasonable benefit/risk ratio and effective for their intended use. The salts may be prepared during the final isolation and purification of the compounds or separately by reacting, for example, an amino group of the compounds with a suitable acid. For example, a compound may be dissolved in a suitable solvent, such as, but not limited to, methanol or water and treated with at least one equivalent of an acid. Basic addition salts may be prepared during the final isolation and purification of the disclosed compounds by reaction of an acidic group (e.g., a carboxyl group) with a suitable base such as the hydroxide, carbonate, or bicarbonate of a cation. The resulting salt may then be precipitated and isolated by filtration and dried under reduced pressure. Alternatively, the solvent and any excess acid may be removed under reduced pressure to provide a salt.

In some embodiments, the compounds provided herein are substantially purified. In some embodiments, the compound is prepared by a process comprising precipitating the compound from a solution comprising the compound, or its salt, and a solvent. In some embodiments, the process comprises drying the precipitated compound. In some embodiments, the precipitated compound is reduced (e.g., ground, collided, or tumbled) to particles.

In some embodiments, compositions are provided comprising one or more compounds described herein. In some embodiments, the compositions are pharmaceutical compositions. The compositions may further comprise a pharmaceutically acceptable carrier.

In some embodiments, the compound is present in the composition in an amount of at least about 90% by weight.

In some embodiments, the composition is a pharmaceutical composition consisting essentially of the compound.

Pharmaceutical compositions for use in accordance with the present disclosure may be formulated in a manner using one or more physiologically acceptable carriers or excipients suitable for a particular route of administration. Thus, the compounds and their physiologically acceptable salts and solvates may be formulated for administration by, for example, solid dosing, eye drop, in a topical oil-based formulation, injection (including injection of a drug-eluting device either into the body as a whole, or into specific tissues of the eye), inhalation (oral or nasal), implants, or oral, buccal, parenteral or rectal administration. Intranasal drug administration may permit bypass of the blood-brain barrier, and may permit delivery to the central nervous system, via the olfactory pathway (Bulbus olfactorius) or via the respiratory pathway (Brainstem, pons). Techniques and formulations may generally be found in “Remington's Pharmaceutical Sciences,” (Meade Publishing Co., Easton, PA).

### Synthesis

In some embodiments, the N-substituted C₆ cyclyl carboxamide compounds provided herein are synthesized by condensation of a carboxylic acid or an acid chloride with an amine in order to form the desired compound.

In some embodiments, the compounds described herein may be prepared according to the following scheme:

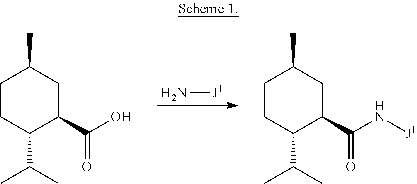

In some embodiments, the compounds described herein may be prepared according to the following scheme:

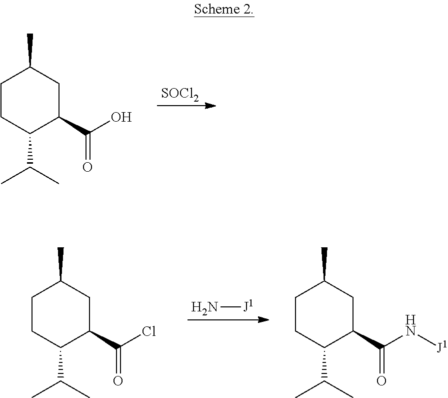

In some embodiments, the compounds described herein may be prepared according to the following scheme:

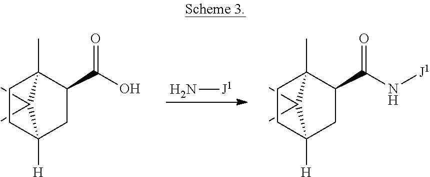

In some embodiments, the compounds described herein may be prepared according to the following scheme:

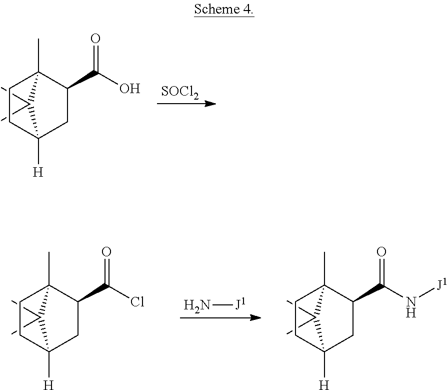

In some embodiments, the compounds described herein may be prepared according to the following scheme:

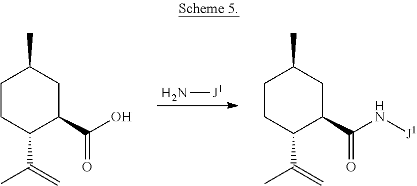

In some embodiments, the compounds described herein may be prepared according to the following scheme:

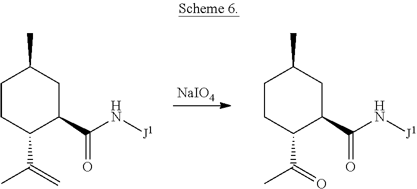

Although J¹-NH₂ is shown in the schemes above, a secondary amine, such as a compound comprising a secondary amine, may be used alternatively under similar reaction conditions. In some embodiments, the secondary amine comprises an aziridinyl, azetidinyl, pyrrolidinyl, piperidinyl, azepanyl, azocanyl, azonanyl, morpholinyl, thiomorpholinyl, piperazinyl, oxazepanyl, or diazepanyl ring.

Thus, in some embodiments, the compounds described herein may be prepared by a process comprising a condensation reaction that includes contacting

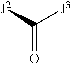

with

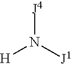

wherein
J¹ is as defined herein;
J² is as defined herein;
J³ is OH or Cl; and
J⁴ is as defined herein;
such that the compound,

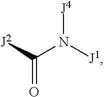

or a salt thereof, is formed.

In some embodiments, the compounds are synthesized by condensation of the carboxylic acid (e.g., (1R,2S,5R)-2-isopropyl-5-methylcyclohexane-1-carboxylic acid or (1S,2S,4R)-1,7,7-trimethylbicyclo[2.2.1]heptane-2-carboxylic acid) or the acid chloride (e.g., (1R,2S,5R)-2-isopropyl-5-methylcyclohexane-1-carbonyl chloride or (1S,2S,4R)-1,7,7-trimethylbicyclo[2.2.1]heptane-2-carbonyl chloride) with the amine R—NH₂.

In some embodiments of these synthetic methods, R is selected from the corresponding moiety shown in any one of the compounds described in Table 1, Table 2, Table 3, Table 4, Table 5, or Table 6, or the corresponding moiety of a generic formula provided herein. In some embodiments, the condensation reaction proceeds without a coupling agent, but may proceed by heating the reaction mixture, for example above about 100° C. In some embodiments, the condensation reaction is facilitated by one or more of a coupling agent. In some embodiments, the coupling agent is, independently, a metal chloride, including without limitation, TiCl₄, or a carbodiimide or its salt, including without limitation, dicyclohexylcarbodiimide (DCC), diisopropylcarbodiimide (DIC), or 1-ethyl-3-(3-dimethylaminopropyl)carbodiimide (EDC). In some embodiments, the condensation further includes one or more of a racemization suppressing additive. In some embodiments, the racemization suppressing additive is, independently, a triazole, including without limitation, 1-hydroxy-benzotriazole (HOBt), or 1-hydroxy-7-aza-benzotriazole (HOAt), ethyl cyanohydroxyiminoacetate, or N-hydroxysuccinimide.

Using substantially the same procedures described above and as in the Examples below, with appropriate modification, the compounds provided herein are prepared.

### Methods

The compounds described herein are useful in modulating the action of ion channels. Accordingly, in some embodiments, the compounds described herein are useful in methods of treating diseases or conditions influenced by ion channels. For example, the above compounds and compositions may be used to modulate the action of ion channels in vitro (e.g., in a cell), or in vivo.

Generally, the compounds provided herein are useful in methods comprising administration of the compound to a subject in need thereof. In some embodiments, the compounds are selected from any of the formulae described herein or from any of the compounds described in the Tables provided herein, and may be provided in neutral form or as a salt of the compound.

In some embodiments, methods are provided for activating an ion channel comprising applying to a medium such as an assay medium or contacting with a cell either in a cell in vitro or in a cell in a living body in vivo an effective amount of a compound as disclosed herein. In one embodiment the ion channel activated or inhibited is TRPM8. TRPM8 modulators or activators are useful in treating various TRPM8-associated diseases or disorders. Examples of TRPM8-associated diseases include diseases or conditions involving the sensation of coolness or dryness. Further examples of TRPM8-associated diseases include dry eye, ocular pain, ocular discomfort, ocular irritation, non-infectious uveitis, and the like. In some embodiments, the disease is dry eye.

In some embodiments, provided herein are methods of ion channel activation in a cell, a tissue, or a subject, such as a human, comprising contacting the cell with an amount of one or more of the compounds of the present disclosure effective to activate the ion channel. In some embodiments, the compounds are administered in a pharmaceutically acceptable composition, such as in or with a pharmaceutically acceptable carrier.

In some embodiments, the compounds of the present disclosure are used in methods for modulating the action of an ion channel in a cell comprising contacting the cell with an amount of one or more compounds of the present disclosure effective to modulate the action of the ion channel in the cell. In some embodiments, the compounds of the present disclosure are administered in a pharmaceutically acceptable composition, such as in or with a pharmaceutically acceptable carrier.

Treatment of diseases or conditions for which the compounds of the present disclosure may be useful includes diseases or conditions associated with ion channel activity or diseases or conditions affected by ion channels. In some embodiments, these types of diseases include inflammatory diseases, ocular inflammatory conditions such as non-infectious uveitis, chorioretinitis, iritis, sterile conjunctivitis, keratitis, episcleritis, dry eye diseases, meibomian gland dysfunction (MGD), allergic conjunctivitis, injury-related ocular inflammation, dry eye syndrome, Primary and Secondary Sjögren's syndrome, redness, blepharitis, keratoconjunctivitis sicca, ocular hyperemia, inflammatory eye diseases, dermatological disorders, respiratory indications, or cancers (e.g., prostate cancers).

In some embodiments, the compounds of the present disclosure may be administered in conjunction with one or more additional therapeutic agents. Suitable classes of additional therapeutic agents include, but are not limited to, ROCK inhibitors, beta blockers, alpha-agonists, carbonic anhydrase inhibitors, prostaglandin-like compounds, miotic agents, cholinergic agents, epinephrine compounds, neuroprotective agents, or anti-inflammatory agents. Non-limiting examples include corticosteroids, immunosuppressive agents, or JAK inhibitors.

Thus, in some embodiments, provided herein are methods of treating an ocular disorder in a subject in need thereof, comprising administering to the subject a compound, a composition, or a pharmaceutical composition provided herein.

In some embodiments, provided herein are methods of reducing ocular inflammation in a subject in need thereof, comprising administering to the subject a compound, a composition, or a pharmaceutical composition provided herein.

In some embodiments of the methods herein, the ocular disorder is dry eye. In some embodiments, the ocular disorder is meibomian gland dysfunction (MGD). In some embodiments, the ocular disorder is uveitis. In some embodiments, the ocular disorder is blepharitis.

In some embodiments, provided herein are methods of reducing inflammation in a subject in need thereof, comprising administering to the subject a compound, a composition, or a pharmaceutical composition provided herein.

In some embodiments of these aspects, the compound, composition, or pharmaceutical composition is administered topically to an eye of the subject.

### Kits

In some embodiments, the compounds described herein may be formulated for ophthalmic use. In some embodiments, the compounds described herein may be formulated as a cream, ointment, or gel for topical administration to the area around a subject's eye, or as a liquid drop for ocular topical instillation. In some embodiments, provided herein are packaged formulations, comprising a container holding a therapeutically effective amount of at least one formulation described herein, and instructions for using the at least one formulation in accordance with one or more of the methods provided herein.

The present formulations and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (e.g., Irgacure 184, 2959, e.g., 1-[4-(2-hydroxyethoxy)-phenyl]-2-hydroxy-2-methyl-1-propane-1-one), preferably water-soluble initiators (e.g. Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (e.g., by the addition of specific product information leaflets) into suitable containers (boxes, etc.).

According to further embodiments, the present formulations can also be provided in kit form combined with other components necessary for administration of the formulation to the patient. For example, disclosed kits, such as for use in the treatment of an eye disease, can further comprise, for example, administration materials including spatulas, rulers, and the like.

The kits are designed in various forms based on the specific deficiencies they are designed to treat.

The formulations provided herein may be prepared and placed in a container for storage at ambient or elevated temperature. When the formulations are stored in a polyolefin plastic container as compared to a polyvinyl chloride plastic container, discoloration of the formulations may be reduced. Without wishing to be bound by theory, the container may reduce exposure of the container's contents to electromagnetic radiation, whether visible light (e.g., having a wavelength of about 380-780 nm) or ultraviolet (UV) light (e.g., having a wavelength of about 190-320 nm (UV B light) or about 320-380 nm (UV A light)). Some containers also include the capacity to reduce adherence or adsorption of the active agent to the surface of the container. Some containers also include the capacity to reduce exposure of the container's contents to infrared light, or a second component with such a capacity. The containers that may be used include those made from a polyolefin such as polyethylene, polypropylene, polyethylene terephthalate, polycarbonate, polymethyl pentene, polybutene, or a combination thereof, especially polyethylene, polypropylene, or a combination thereof. In some embodiments, the container is a glass container, and the user scoops the contents out of the container with a measured spatula, scoop, or the like. In some embodiments the container is malleable, which permits the user to squeeze the contents out of the container. The container may further be disposed within a second container, for example, a paper, cardboard, paperboard, metallic film, or foil, or a combination thereof, container to further reduce exposure of the container's contents to UV, visible, or infrared light. The formulations provided herein benefit from reduced discoloration, decomposition, or both during storage in such containers. The formulations provided herein may need storage lasting up to, or longer than, three months; in some cases up to, or longer than, one year. The containers may be in any form suitable to contain the contents; for example, a bag, a bottle, a tube, or a box. In some embodiments the container is a single use blow-fill-seal tube. In some embodiments, the container is a multi-dose bottle.

The following examples further illustrate aspects of the present disclosure. However, they are in no way a limitation of the teachings or disclosure as described herein.

### Examples

### Example 1. Compound Synthesis

(1R,2S,5R)-2-isopropyl-5-methyl-N-(3-(methylsulfonyl)cyclopentyl)cyclohexane-1-carboxamide is prepared according to the following scheme:

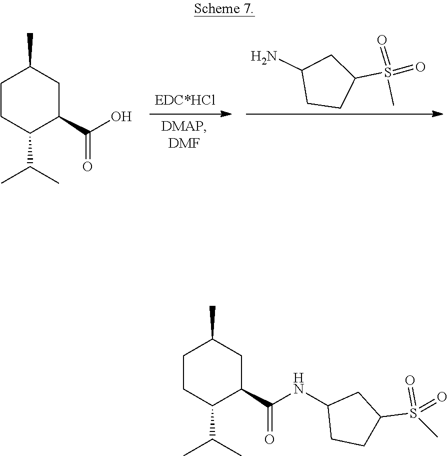

To (1R,2S,5R)-2-isopropyl-5-methylcyclohexane-1-carboxylic acid (38 mg, 0.21 mmol) in DMF (0.8 mL) was added 3-(methylsulfonyl)cyclopentan-1-amine (50 mg, 0.25 mmol), N-(3-Dimethylaminopropyl)-N-ethylcarbodiimide hydrochloride (EDC·HCl, 48 mg, 0.25 mmol) and DMAP (31 mg, 0.25 mmol) and the solution was stirred at room temperature overnight. The compound was poured into EtOAc and extracted with HCl (1N), dried (Na₂SO₄), filtered and evaporated. Automated column (4 g Teledyne cartridge) 0-100% EtOAc-Hexanes gave pure (1R,2S,5R)-2-isopropyl-5-methyl-N-(3-(methylsulfonyl)cyclopentyl)cyclohexane-1-carboxamide (42 mg, 60%).

### Example 2. Compound Synthesis

(1R,2S,5R)-2-isopropyl-N-(6-methoxy-5-methylpyridin-3-yl)-5-methylcyclohexane-1-carboxamide is prepared according to the following scheme:

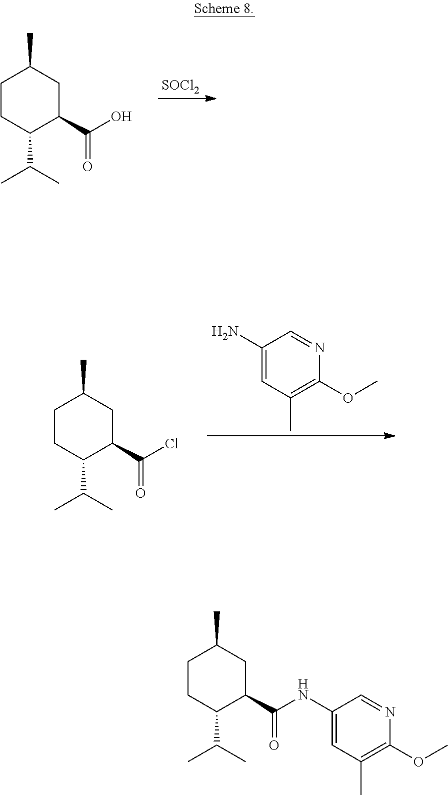

(1R,2S,5R)-2-isopropyl-5-methylcyclohexane-1-carboxylic acid (300 mg, 1.6 mmol) was added thionyl chloride (1.5 mL) and the solution was stirred at reflux 85-87° C. After 1.5 hours, the solution was cooled and the residual thionyl chloride was evaporated to give (1R,2S,5R)-2-isopropyl-5-methylcyclohexane-1-carbonyl chloride (319 mg, >95%).

To 6-methoxy-5-methylpyridin-3-amine (72 mg, 0.52 mmol) in CH₂Cl₂ (0.5 mL) was added pyridine (104 μL, 1.3 mmol) and the solution was cooled to 0° C. Then (1R,2S,5R)-2-isopropyl-5-methylcyclohexane-1-carbonyl chloride (115 mg, 0.57 mmol) was added and the solution was warmed to room temperature and stirred. After 1.3 hours the compound was poured into EtOAc and extracted with HCl (1N) and then with NaHCO₃(saturated). The organic layer was dried (Na₂SO₄), filtered and evaporated. Automatic column (4 g Teledyne cartridge, 0-15-20% EtOAc-Hexane) gave pure (1R,2S,5R)-2-isopropyl-N-(6-methoxy-5-methylpyridin-3-yl)-5-methylcyclohexane-1-carboxamide (86 mg, 54%).

### Example 3. Compound Synthesis

(1S,2S,4R)-N-(4-methoxyphenyl)-1,7,7-trimethylbicyclo[2.2.1]heptane-2-carboxamide is prepared according to the following scheme:

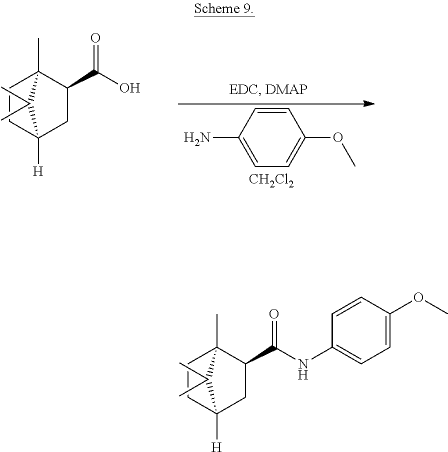

To (1S,2S,4R)-1,7,7-trimethylbicyclo[2.2.1]heptane-2-carboxylic acid in CH₂Cl₂ (2 mL) was added EDC·HCl (131 mg, 0.7 mmol), DMAP (83 mg, 0.7 mmol) and 4-methoxyaniline (84 mg, 0.7 mmol) and the solution was stirred overnight at room temperature. The mixture was diluted with CH₂Cl₂ and extracted with HCl (1N), dried (Na₂SO₄), evaporated and filtered. Automated column chromatography (Teledyne 4g cartridge 0-30% EtOAc-Hexanes) gave pure (1S,2S,4R)-N-(4-methoxyphenyl)-1,7,7-trimethylbicyclo[2.2.1]heptane-2-carboxamide (119 mg, 73%)

### Example 4. Compound Synthesis

Isopulegyl carboxylic acid is prepared according to the following scheme:

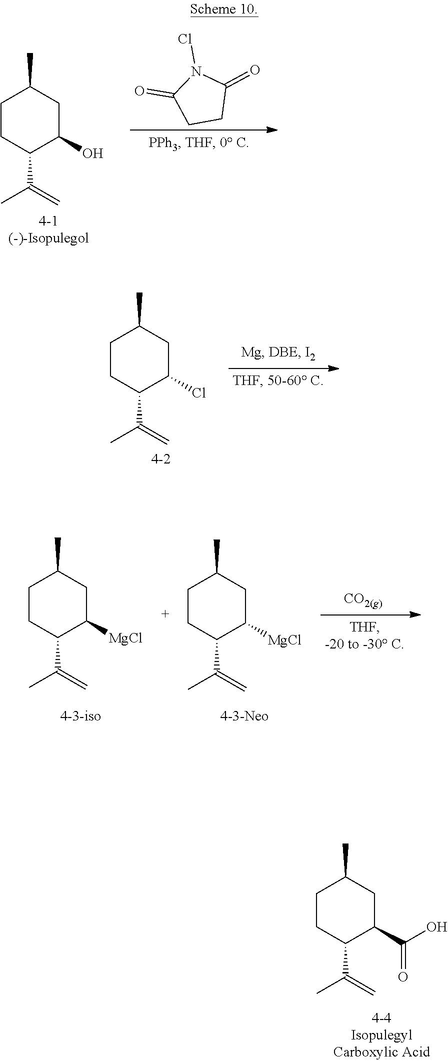

To an ice bath cooled solution of 6.95 g (52 mmol) NCS in 75 mL dry THF under N₂ was slowly added, over the course of an hour via addition funnel, a 20 mL solution of 12.8 g (48.8 mmol) PPh₃. Solution was allowed to warm to room temperature and stir for one hour. After which a 75 mL solution of 5.016 g (32.5 mmol) (−)-isopulegol (4-1) in dry THF was added dropwise. Reaction mixture was stirred at room temperature under N₂ for four days, during which it turns a very dark maroon color. Once the TLC indicates no starting material present, the reaction mixture was concentrated, followed by the addition of 200 mL pentane. Solids were filtered out over a pad of celite and crude material purified by distillation to afford 5.1 g of 4-2 in 91% yield. TLC: 100% pentane.

In an oven-dried round bottom flask, 82.7 mg (34.0 mmol) freshly crushed magnesium turnings were activated with 12 and DBE at 50° C., then cooled. In dry THF, 4.2 g (24.3 mmol) chloride 4-2 was added portion wise to activated magnesium at 50-60° C. Bubbling should occur, as well as darkening of magnesium. Reaction was allowed to stir at 50° C. overnight under N₂. Reaction mixture was cooled and used immediately.

At room temperature, CO_{2(g)} was bubbled into reaction mixture for 3 hours. Reaction mixture was quenched with 1M KHSO₄ and extracted with ethyl acetate three times. The combined organic layers were extracted with 1M NaOH, which was then acidified with 1M HCl. This aqueous layer was extracted with ethyl acetate three times. Followed by DCM twice. The combined organic layers were then washed with brine and dried. Obtained 1.189 g Isopulegyl Carboxylic Acid 4-4 after column chromatography in 27% yield. Use pentane instead of heptane. TLC: (9:1:1) pentane:ethyl acetate:acetic acid.

### Example 5. Compound Synthesis

(1R,2R,5R)-2-acetyl-N-(4-methoxyphenyl)-5-methylcyclohexane-1-carboxamide is prepared according to the following scheme:

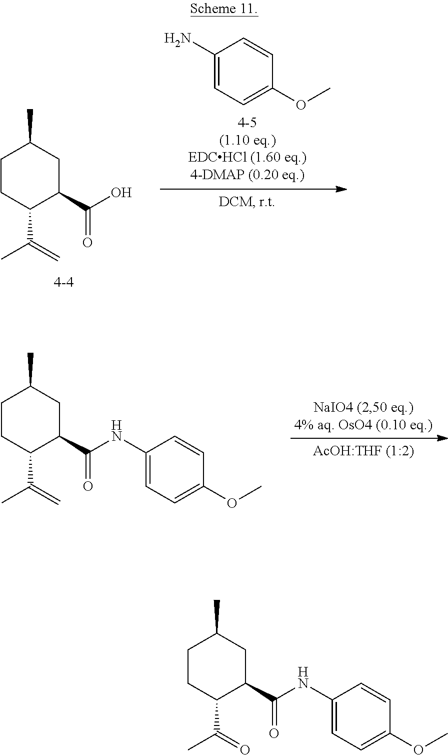

To a solution (1R,2R,5R)-5-methyl-2-(prop-1-en-2-yl)cyclohexane-1-carboxylic acid (4-4) (50 mg, 0.27 mmol, 1.0 eq) in DCM was charged p-anisidine (41 mg, 0.29 mmol, 1.1 eq., 4-5), EDC HCl (83 mg, 0.43 mmol, 1.6 eq.), and 4-DMAP (6.6 mg, 0.05 mmol, 0.20 eq.). The solution was stirred at room temperature. After six hours, the reaction mixture was poured into aqueous sodium bicarbonate and ethyl acetate. The layers were separated, and the aqueous layer was thrice extracted with ethyl acetate. The combined organics were dried over magnesium sulfate, filtered, and evaporated. The crude residue was purified by column chromatography (hexanes:ethyl acetate) to afford (1R,2R,5R)-N-(4-methoxyphenyl)-5-methyl-2-(prop-1-en-2-yl)cyclohexane-1-carboxamide in 92% yield.

Sodium periodate (61 mg, 0.29 mmol, 2.5 eq.) was dissolved in acetic acid (0.5 mL), water (0.5 mL), and THF (0.5 mL). (1R,2R,5R)-N-(4-methoxyphenyl)-5-methyl-2-(prop-1-en-2-yl)cyclohexane-1-carboxamide (33 mg, 0.11 mmol, 1 eq.) was dissolved in 1 mL THF and added to the sodium periodate solution. Osmium tetroxide (4% aq., 0.08 mL) was added, and the reaction stirred at room temperature for four hours. The reaction mixture was poured into water and thrice extracted with ethyl acetate. The combined organics were dried over magnesium sulfate, filtered, and then evaporated. The crude residue was purified by column chromatography (hexanes:ethyl acetate) to afford (1R,2R,5R)-2-acetyl-N-(4-methoxyphenyl)-5-methylcyclohexane-1-carboxamide in 62% yield.

### Example 6. TRPM8 and TRPV1 Assays

Compounds can be tested using the commercially available kit from Eurofins “TRPM8 Human Transient Potential Ion Channel Cell Based Agonist Calcium Flux Assay, Cerep” item #3316, or using the assay, referenced by Eurofins, and described in Behrendt et al. “Characterization of the mouse cold-menthol receptor TRPM8 and vanilloid receptor type-1 VR1 using a fluorometric imaging plate reader (FLIPR) assay,” Br J Pharmacol. 2004 Feb;141(4):737-45, which are each incorporated herein by reference. For reference, the TRPM8 EC₅₀ values of certain TRPM8 agonists are described by Behrendt et al. as having the following relative potencies: icilin (0.2±0.1 μM)>FrescolatML (3.3±1.5 μM)>WS-3 (3.7±1.7 μM)>(−)menthol (4.1±1.3 μM)>frescolatMAG (4.8±1.1 μM)>cooling agent 10 (6±2.2 μM)>(+)menthol (14.4±1.3 μM)>PMD38 (31±1.1 μM)>WS-23 (44±7.3 μM)>Coolact P (66±20 μM)>geraniol (5.9±1.6 mM)>linalool (6.7±2.0 mM)>eucalyptol (7.7±2.0 mM)>hydroxycitronellal (19.6±2.2 mM). 4. For reference, the IC₅₀ values of certain TRPV1 antagonists to block the response of TRPM8 to menthol are described by Behrendt et al. as: BCTC (4-(3-Chloro-2-pyridinyl)-N-[4-(1,1-dimethylethyl)phenyl]-1-piperazinecarboxamide; 0.8±1.0 μM), thio-BCTC (3.5±1.1 μM), and capsazepine (18±1.1 μM).

In brief, compounds provided herein are diluted to the appropriate concentration levels, typically log or half-log dilutions with a 1 μM center concentration with the control activator ligand of icilin. The assay is done at room temperature and measures calcium flux. The website reports that dl-menthol has a TRPM8 EC₅₀ of 6000 nM and icilin has a TRPM8 EC₅₀ of 28 nM. Activities of certain compounds described herein are shown in Table 7, Table 8, Table 9, and Table 10.

TABLE 7

| | | |
|---|---|---|
| Compound Number | TRPM8 EC₅₀ | Percent Agonist |

| 1 | 24 | nM | 100 |
|---|---|---|---|
| 3 | <500 | nM | >75 |
| 5 | <500 | nM | >75 |
| 7 | ND | | |
| 9 | <5000 | nM | >75 |
| 11 | <500 | nM | >75 |
| 13 | ND | | ND |
| 15 | <100 | nM | >75 |
| 17 | <100 | nM | >75 |
| 19 | <500 | nM | >75 |
| 21 | <100 | nM | >75 |
| 23 | <500 | nM | >75 |
| 24 | <500 | nM | >100 |
| 20 | <500 | nM | >100 |
| 22 | <500 | nM | >100 |
| 18 | <100 | nM | >50 |
| 2 | <500 | nM | >100 |
| 4 | <100 | nM | >50 |
| 6 | <100 | nM | >75 |
| 8 | ND | | |
| 10 | <5000 | nM | >75 |

| | | |
|---|---|---|
| 12 | ND | ND |
| 14 | ND | ND |
| 16 | ND | ND |

TABLE 8

| | | |
|---|---|---|
| Compound Number | TRPM8 EC₅₀ | Percent Agonist |

| 25 | <100 | nM | >75 |
|---|---|---|---|
| 27 | <5000 | nM | >75 |
| 29 | <1000 | nM | >100 |
| 31 | <100 | nM | >75 |
| 33 | <1000 | nM | >100 |
| 35 | <100 | nM | >75 |
| 37 | <1000 | nM | >100 |
| 39 | ND | | |
| 41 | ND | | |
| 43 | <1000 | nM | >75 |
| 45 | <1000 | nM | >100 |
| 47 | <500 | nM | >100 |
| 49 | <500 | nM | >75 |
| 51 | <100 | nM | >75 |
| 53 | >10 | μM | |
| 55 | <100 | nM | >75 |
| 57 | <1000 | nM | >75 |
| 59 | <1000 | nM | >75 |
| 58 | ND | | |
| 46 | <500 | nM | >100 |
| 48 | <500 | nM | >50 |
| 50 | <500 | nM | >100 |
| 52 | <1000 | nM | >100 |
| 54 | >10 | μM | >75 |
| 56 | <10000 | nM | >75 |
| 36 | <10000 | nM | >75 |
| 38 | <500 | nM | >75 |
| 40 | ND | | |
| 42 | ND | | |
| 44 | <1000 | nM | >75 |
| 32 | <2000 | nM | >75 |
| 34 | <1000 | nM | >75 |
| 26 | <1000 | nM | >75 |
| 28 | <10000 | nM | >75 |
| 30 | >10 | μM | |

TABLE 9

| | | |
|---|---|---|
| Compound Number | TRPM8 EC₅₀ | Percent Agonist |

| 60 | ND | | |
|---|---|---|---|
| 62 | <500 | nM | >75 |
| 64 | >10 | μM | |
| 66 | ND | | |
| 68 | <7500 | nM | >75 |
| 70 | <1000 | nM | >75 |
| 72 | <5000 | nM | >75 |
| 74 | <100 | nM | >50 |
| 76 | <100 | nM | >50 |
| 78 | <500 | nM | >75 |
| 80 | <1000 | nM | >75 |
| 82 | <500 | nM | >75 |
| 84 | <100 | nM | >75 |
| 86 | <5000 | nM | >100 |
| 88 | <5000 | nM | >35 |
| 73 | <100 | nM | >50 |
| 75 | <5000 | nM | >75 |
| 77 | <5000 | nM | >50 |
| 79 | <2000 | nM | >75 |
| 81 | <100 | nM | >100 |
| 83 | <5000 | nM | >75 |
| 85 | | | |
| 87 | | | |
| 71 | | | |
| 61 | <100 | nM | >75 |
| 63 | | | |
| 65 | <1000 | nM | >50 |
| 67 | <100 | nM | >75 |

| | | |
|---|---|---|
| 69 | <10000 | >75 |

TABLE 10

| Compound Number | Structure | TRPM8 EC₅₀ | | Percent Agonist |
|---|---|---|---|---|
| 170 | | <5000 | nM | >75 |
| 172 | | <3000 | nM | >100 |
| 174 | | >10 | μM | |
| 176 | | ND | | ND |
| 178 | | ND | | ND |
| 171 | | >10 | μM | |
| 173 | | >10 | μM | |
| 175 | | ND | | ND |
| 177 | | >10 | μM | |
| 89 | | <1000 | nM | >75 |
| 91 | | <1000 | nM | >75 |
| 93 | | <10000 | nM | >75 |
| 95 | | >10 | μM | |
| 97 | | >10 | μM | |
| 99 | | <10000 | nM | >75 |
| 101 | | <5000 | nM | >75 |
| 103 | | ND | | |
| 105 | | ND | | |
| 104 | | | | |
| 106 | | <10000 | nM | >50 |
| 94 | | <500 | nM | >75 |
| 107 | | <500 | nM | >100 |
| 98 | | <500 | nM | >75 |
| 100 | | >10 | mM | >35 |
| 92 or 102 | 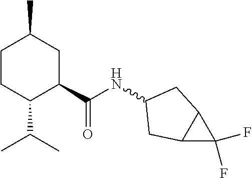 [SMILES: CC(C)[C@@H]1CC[C@@H](C)C[C@H]1C(=O)NC1CC2C(C1)C2(F)F] | <100 | nM | >50 |
| | Diastereomer 1 | | | |
| | (chromatographically resolved) | | | |
| 102 or 92 | 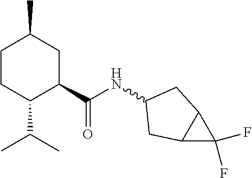 [SMILES: CC(C)[C@@H]1CC[C@@H](C)C[C@H]1C(=O)NC1CC2C(C1)C2(F)F] | <500 | nM | >75 |
| | Diastereomer 2 | | | |
| | (chromatographically resolved) | | | |
| 90 | | | | |
| 109 | | ND | | ND |
| 108 | | >10 | μM | >5 |

### Example 7. Topical Pharmaceutical Compositions

Topical pharmaceutical compositions for treating inflammation are prepared by conventional methods and formulated as shown in Table 11.

TABLE 11

| Ingredient | Amount (wt %) |
|---|---|
| N-Substituted C₆ Cyclyl Carboxamide Compound | 0.30 |
| Citric Acid | 0.1 |
| Hydroxypropyl methylcellulose | 0.3 |
| Sodium Chloride | 0.77 |
| Potassium chloride | 0.12 |
| Disodium EDTA | 0.05 |
| Benzalkonium chloride | 0.01 |
| HCl and/or NaOH | pH 4.5-6.5 |
| Purified water | q.s. to 100% |

When the composition is topically administered to the eyes once daily, the above composition decreases ocular inflammation in a subject suffering from MGD or DED.

### Example 8. Topical Pharmaceutical Compositions

Topical pharmaceutical compositions for treating inflammation are prepared by conventional methods and formulated as shown in Table 12.

TABLE 12

| Ingredient | Amount (wt %) |
|---|---|
| N-Substituted C₆ cyclyl Carboxamide Compound | 0.3 |
| Citric acid | 0.067 |
| Sodium Citrate Dihydrate | 0.48 |
| Sodium Chloride | 0.70 |
| HCl and/or NaOH | pH 6.0 |
| Purified water | q.s. to 100% |

When the composition is topically administered to the eyes once daily (e.g., via topical instillation), the above composition decreases ocular inflammation in a subject suffering from Meibomian Gland Dysfunction (MGD) or Dry Eye Disease (DED).

1. A compound, having a formula: 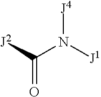
or a pharmaceutically acceptable salt thereof,
wherein
J¹ is C₁₋₁₃H₂₋₁₉N₀₋₂O₀₋₃S₀₋₁B₀₋₁F₀₋₃Cl₀₋₁ comprising 0 to 3 rings and a molecular weight of about 39.0 to about 220.3;
J² is 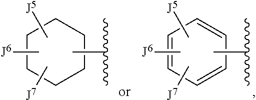
wherein
J⁵ is halo, C₁₋₃ alkyl, C₁₋₃ haloalkyl, OH, NH₂, N(H)Ac, N(H)Boc, N(H)Fmoc, or N(H)Cbz;
J⁶ is H; and
J⁷ is H, propyl, ethyl-NH₂, ethyl-OH, acetyl, propenyl, propenolyl, methyloxiranyl, or methylenecyclopropanyl;
or J⁶ and J⁷ together form a C₁₋₆ alkylenyl bridge; and
J⁴ is H, C₁₋₆ heteroalkyl, C₆₋₁₂ aryl, or C(O)—(C₁₋₃ alkylene)-(C₂₋₃ heterocycloalkyl);
or J¹ and J⁴, together with the nitrogen to which they are attached, form an unsubstituted C₂₋₈ heterocycloalkyl, or C₂₋₈ heterocycloalkyl substituted with one or two groups independently selected from C₁₋₆ alkyl or C₁₋₆ haloalkyl, wherein hetero refers to one, two, or three of, independently, N, O, or S.

2. The compound of claim 1, wherein 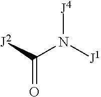
is 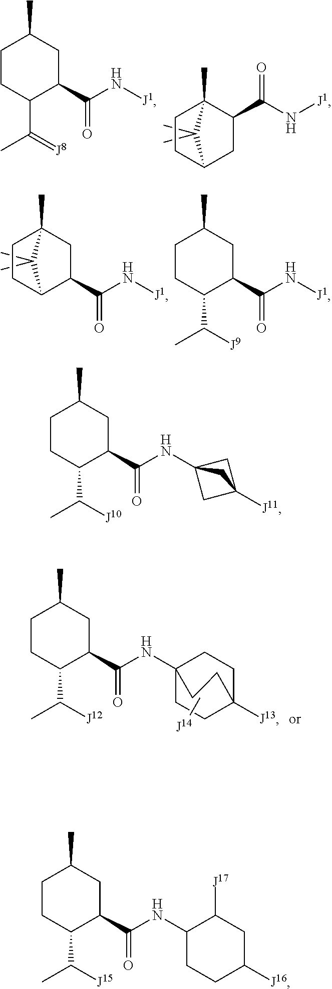
wherein
J⁸ is CH₂ or O;
J⁹ is CH₃, NH₂, or OH;
J¹⁰ is CH₃, NH₂, or OH;
J¹¹ is NH₂, CN, C₁₋₃ alkyl, F, C₁₋₃ haloalkyl, O(C₁₋₃ alkyl), (C₁₋₃ alkylenyl)CN, (C₁₋₃ alkylenyl) OH, (C₁₋₃ alkylenyl)O(C₁₋₃ alkyl), C(O)OH, (C₁₋₃ alkylenyl)C(O)OH, C(O)O(C₁₋₃ alkyl), (C₁₋₃ alkylenyl)C(O)O(C₁₋₃ alkyl), C(O)O(C₁₋₆ alkyl), tetramethyldioxaborolanyl, CO₂NH₂, C(O)N(C₁₋₃ alkyl)O(C₁₋₃ alkyl), N(H)C(O)(C₁₋₃ alkylenyl)OH, or N(H)C(O)O(C₁₋₃ alkyl);
J¹² is CH₃, NH₂, or OH;
J¹³ is O(C₁₋₃ alkyl) or C(O)O(C₁₋₃ alkyl);
J¹⁴ is H;
J¹⁵ is CH₃, NH₂, or OH;
J¹⁶ is H, O(C₁₋₃ alkyl), or O(C₁₋₃ alkylenyl)O(C₁₋₃ alkyl); and
J¹⁷ is H, or forms a C₈₋₁₀ spirocycloalkyl substituted with NH₂ or NHC(O)(C₁₋₃ alkylenyl)NH₂.

3. The compound of claim 2, wherein:
J¹¹ is NH₂, CN, CH₂CH₃, F, CH₂CF₃, OCH₃, OCH₂CH₃, CH₂CN, CH₂OH, CH₂CH₂OH, CH₂OCH₃, C(O)OH, CH₂C(O)OH, C(O)OCH₃, CH₂C(O)OCH₃, C(O)OC(CH₃)₃, tetramethyldioxaborolanyl, CO₂NH₂, C(O)N(CH₃)OCH₃, N(H)C(O)CH₂OH, or N(H)C(O)OCH₃;
J¹³ is OCH₃ or C(O)OCH₃;
J¹⁶ is H, OCH₃, or OCH₂CH₂OCH₃; or
J¹⁷ is H, or forms a spirononanyl substituted with NH₂ or NHC(O)CH₂NH₂.

4. The compound of claim 1, wherein:
J¹ is 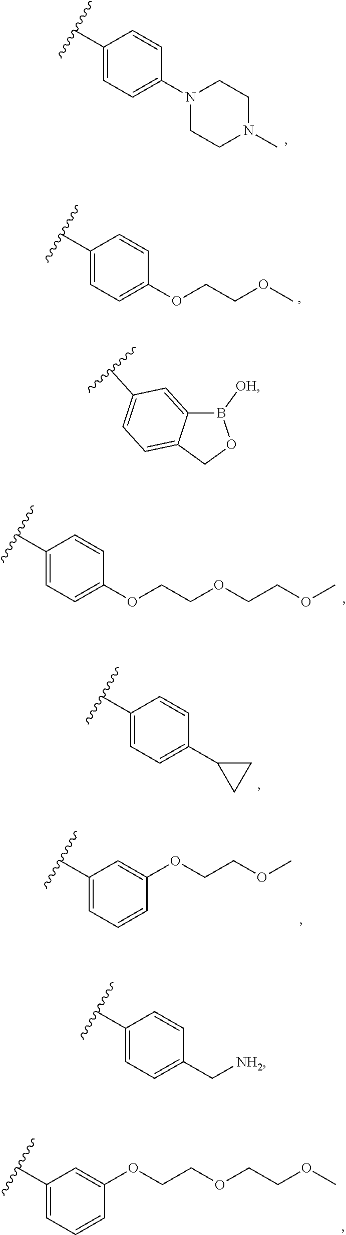 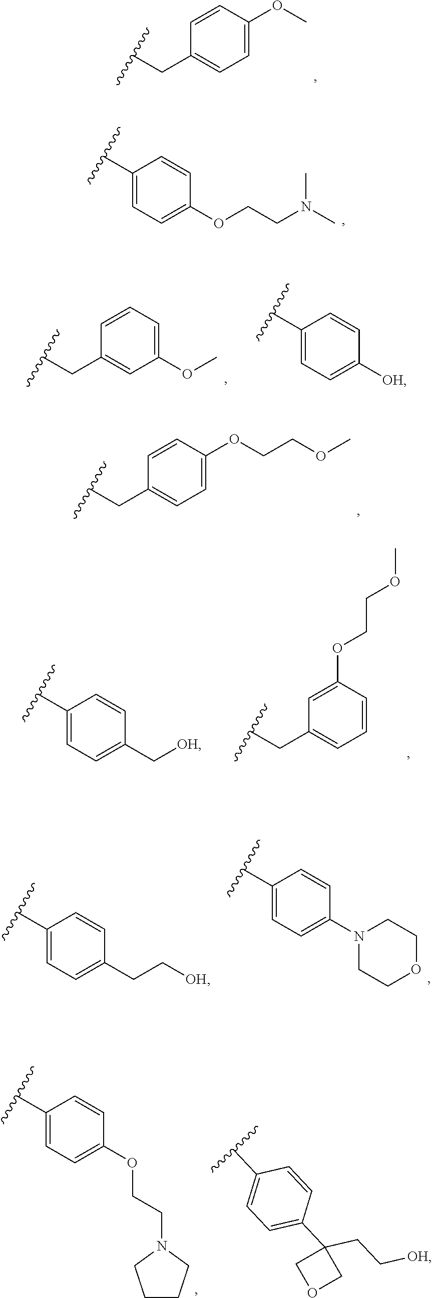 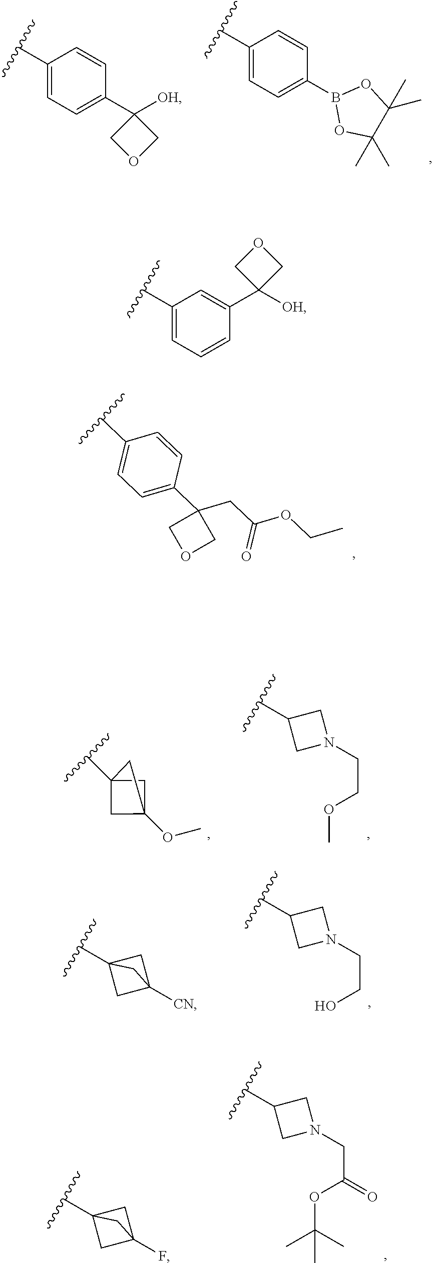 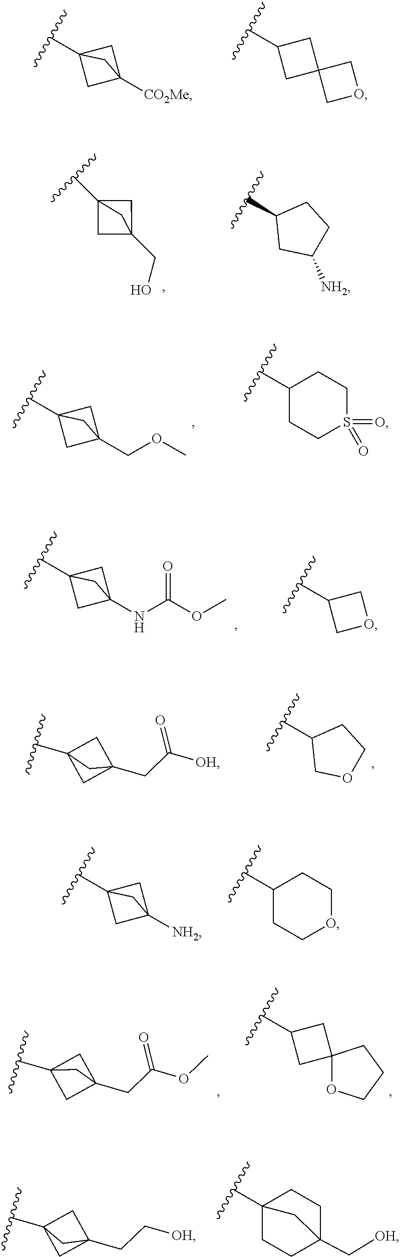 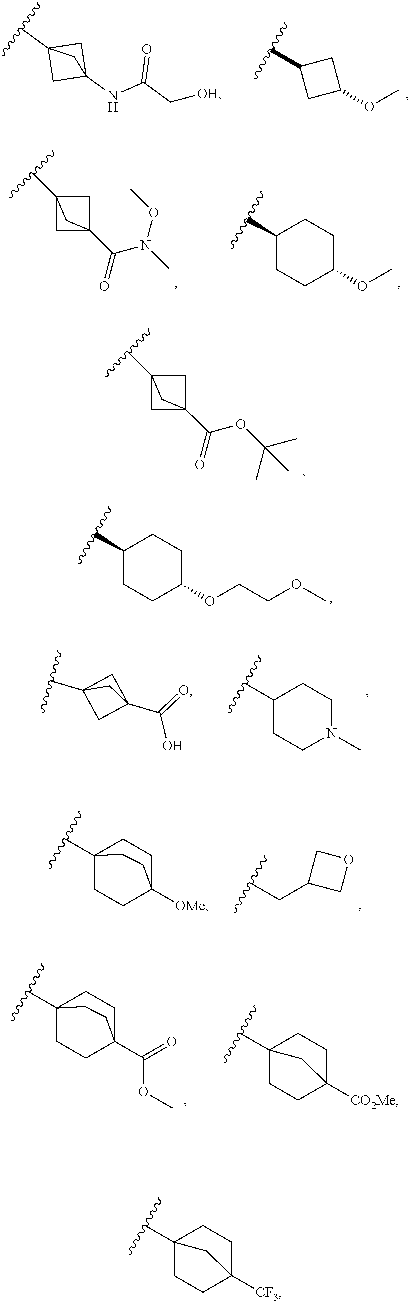 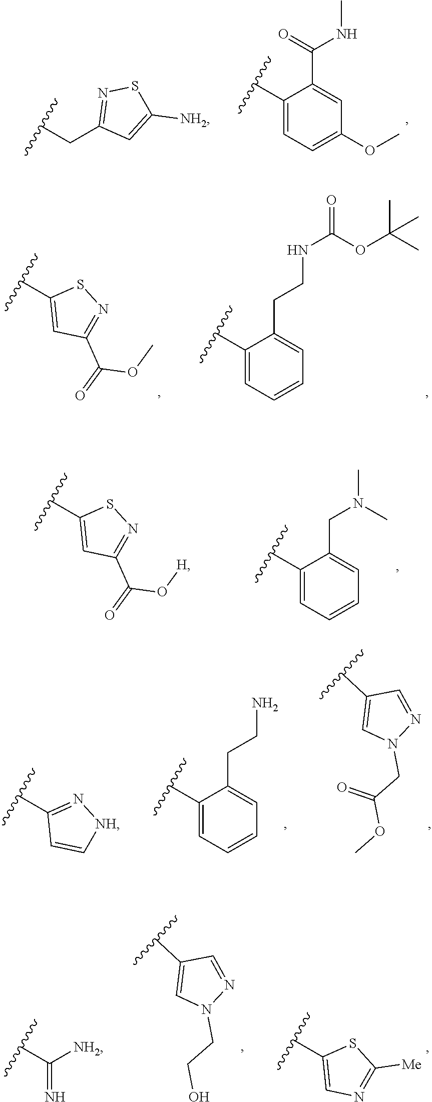 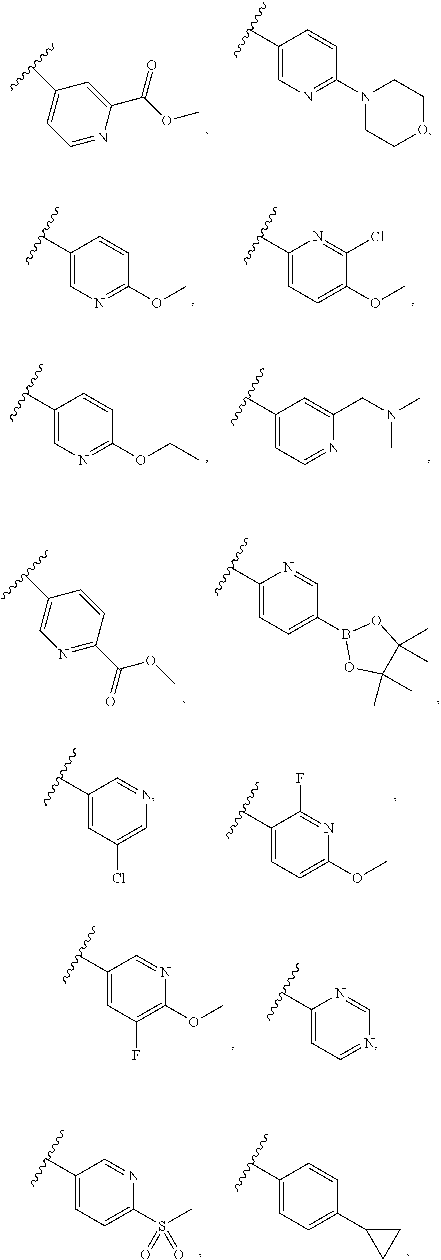 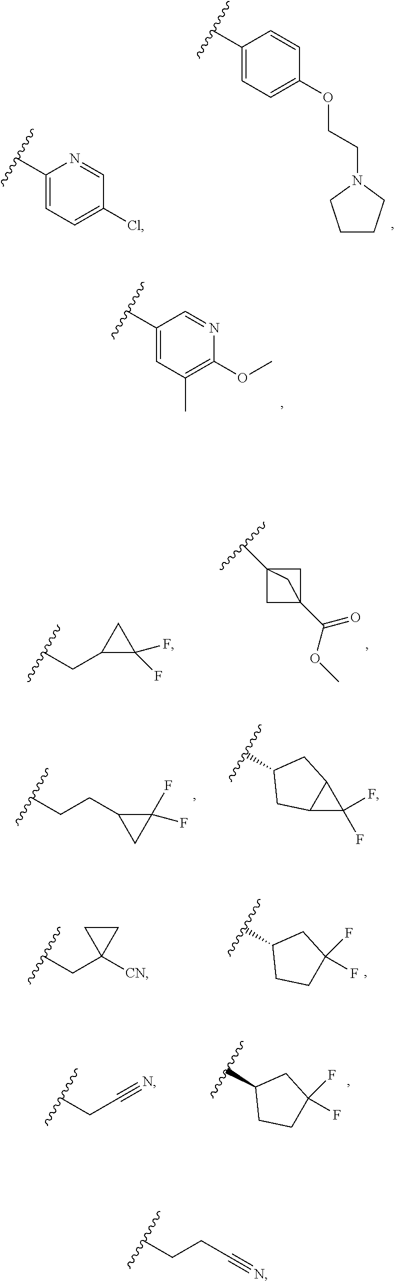 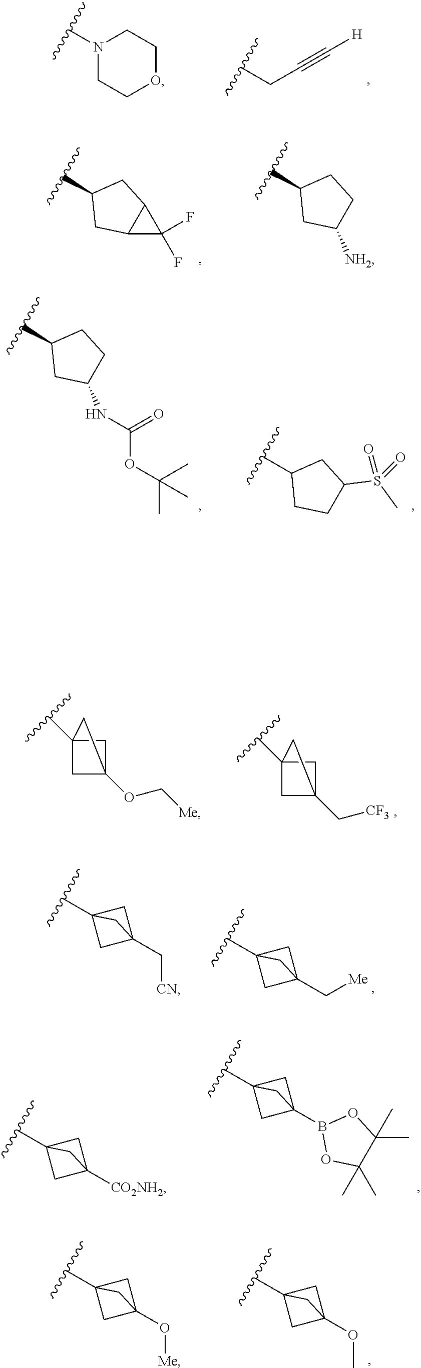 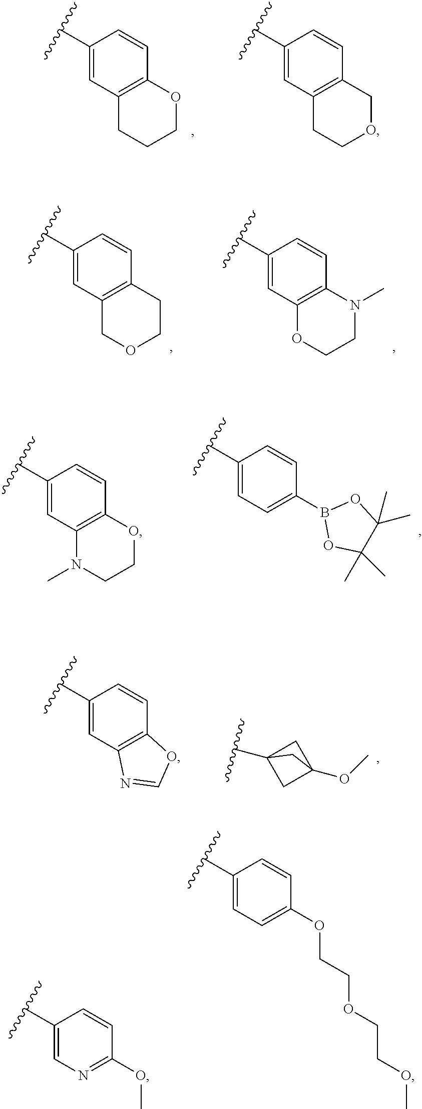 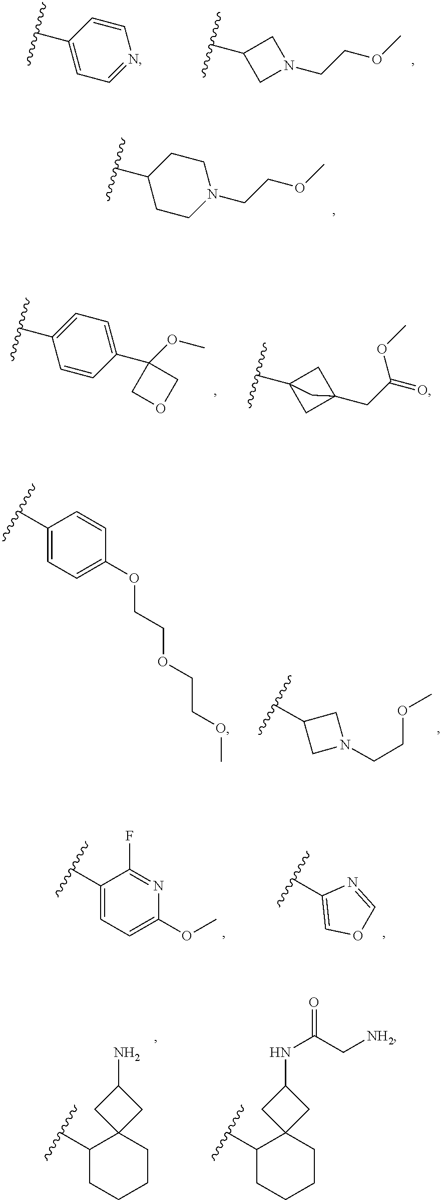 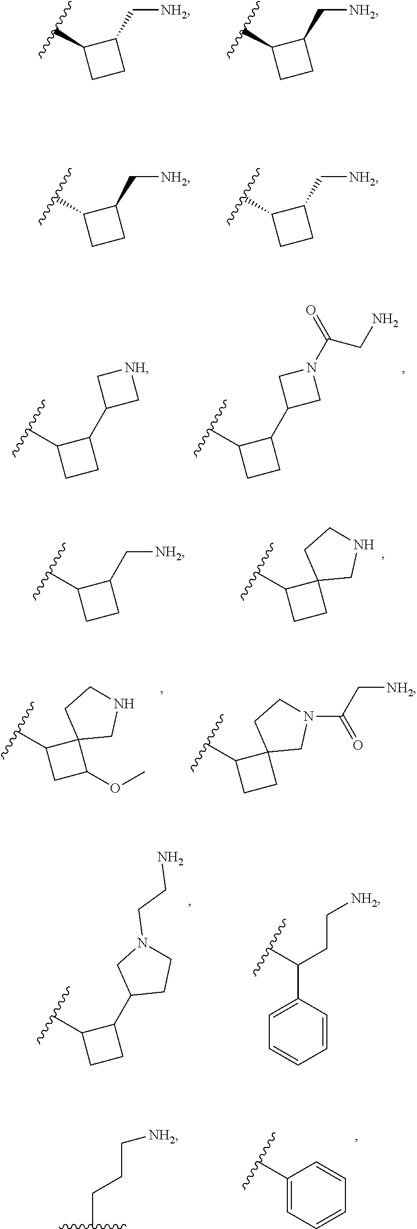 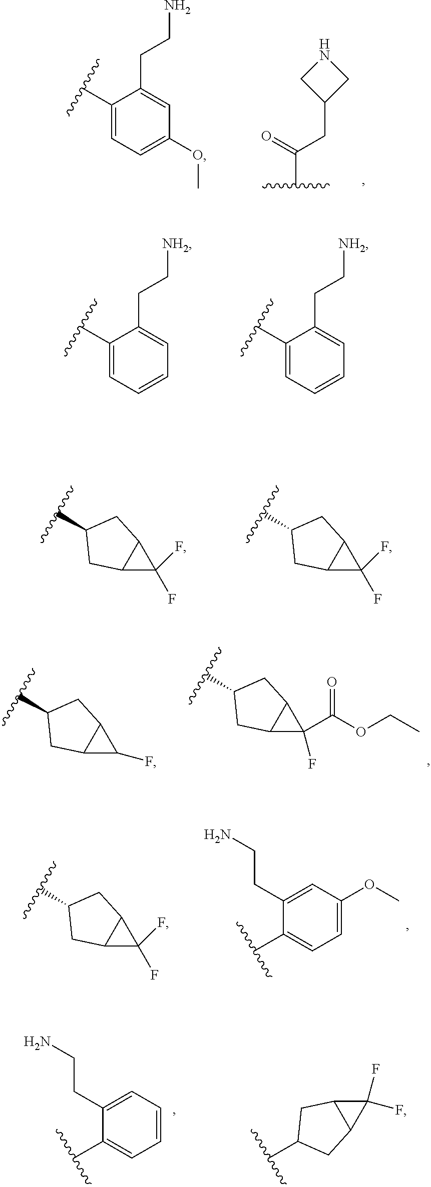 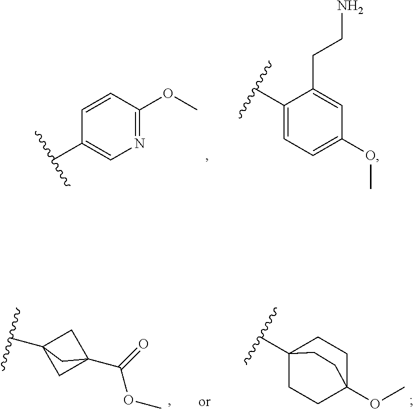
J² is 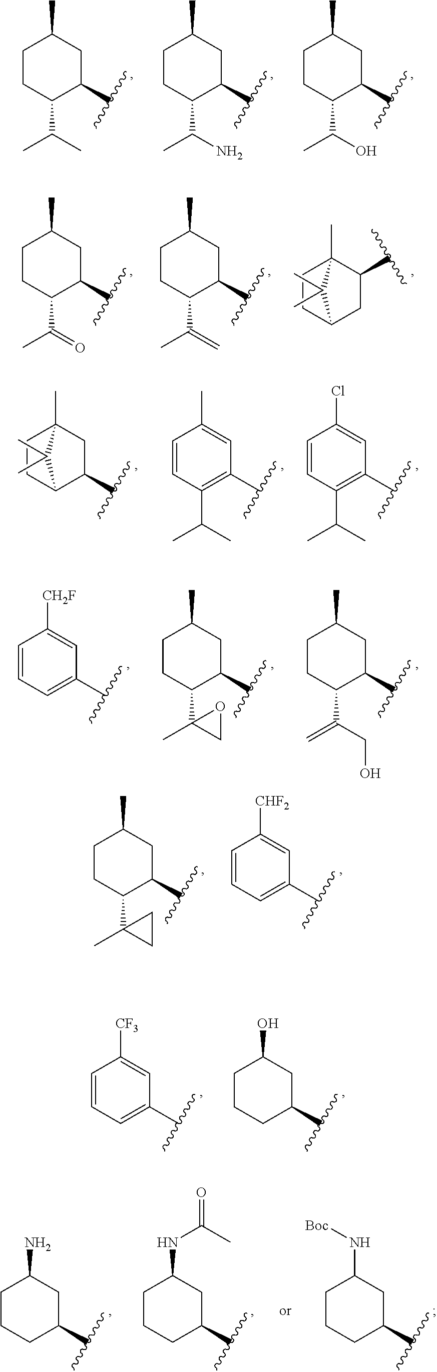
J⁴ is H, 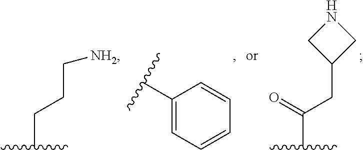
or J¹ and J⁴, together with the nitrogen to which they are attached, form

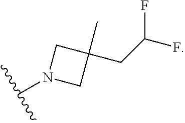 [SMILES: *N1CC(C)(CC(F)F)C1]

5. The compound of claim 1, wherein J¹ comprises 1, 2, or 3 rings, a molecular weight of about 80 to about 200, and at least two atoms selected, independently, from N, O, S, B, F, or Cl.

6-21. (canceled)

22. A compound, selected from: 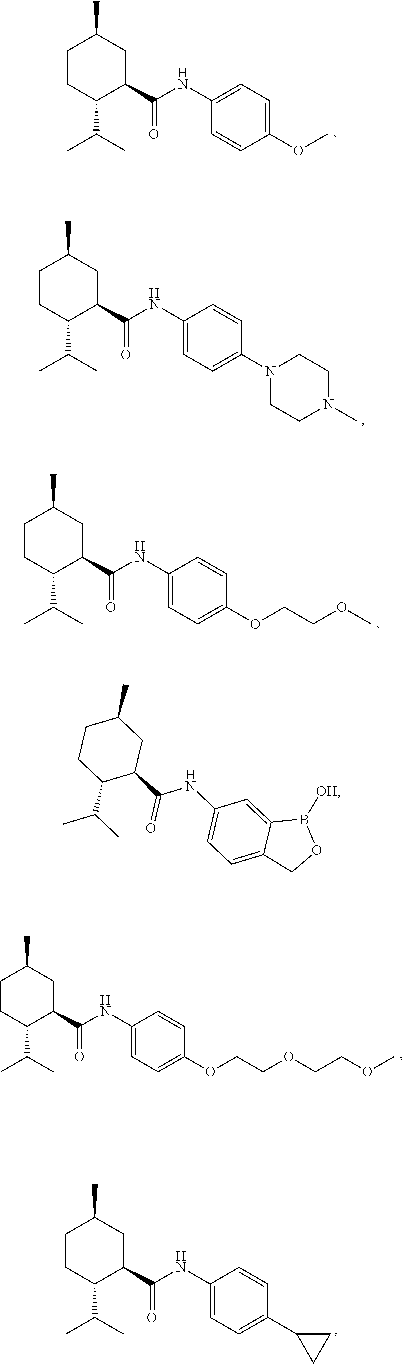 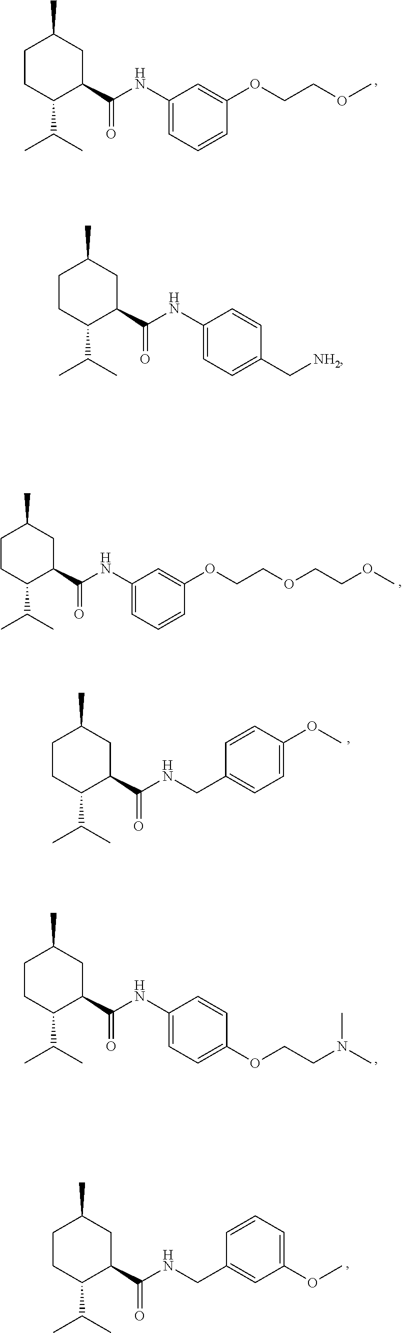 [SMILES: COCCOc1cccc(NC(=O)[C@@H]2C[C@H](C)CC[C@H]2C(C)C)c1 ; CC(C)[C@@H]1CC[C@@H](C)C[C@H]1C(=O)Nc1ccc(CN)cc1 ; COCCOCCOc1cccc(NC(=O)[C@@H]2C[C@H](C)CC[C@H]2C(C)C)c1 ; COc1ccc(CNC(=O)[C@@H]2C[C@H](C)CC[C@H]2C(C)C)cc1 ; CC(C)[C@@H]1CC[C@@H](C)C[C@H]1C(=O)Nc1ccc(OCCN(C)C)cc1 ; COc1cccc(CNC(=O)[C@@H]2C[C@H](C)CC[C@H]2C(C)C)c1] 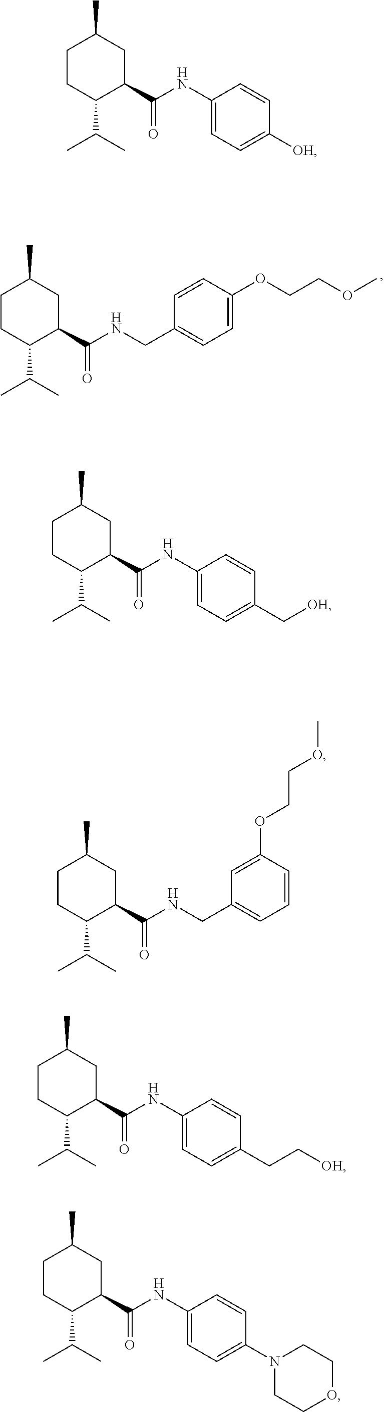 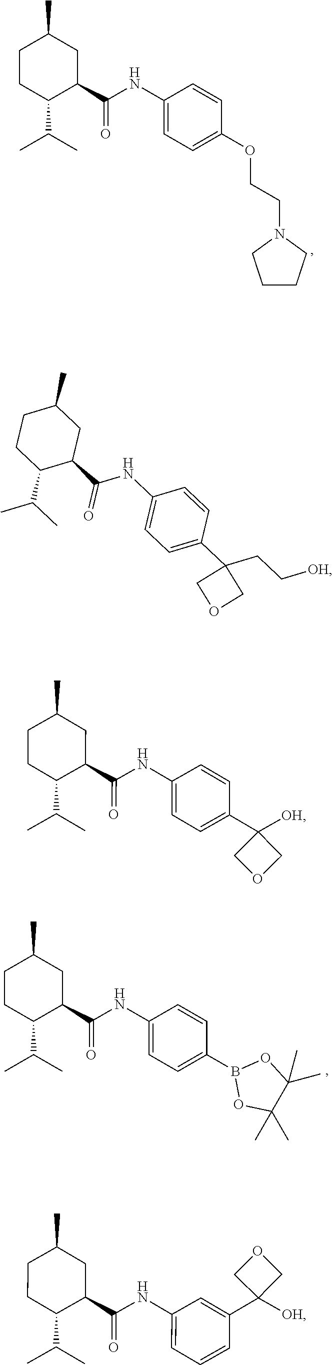 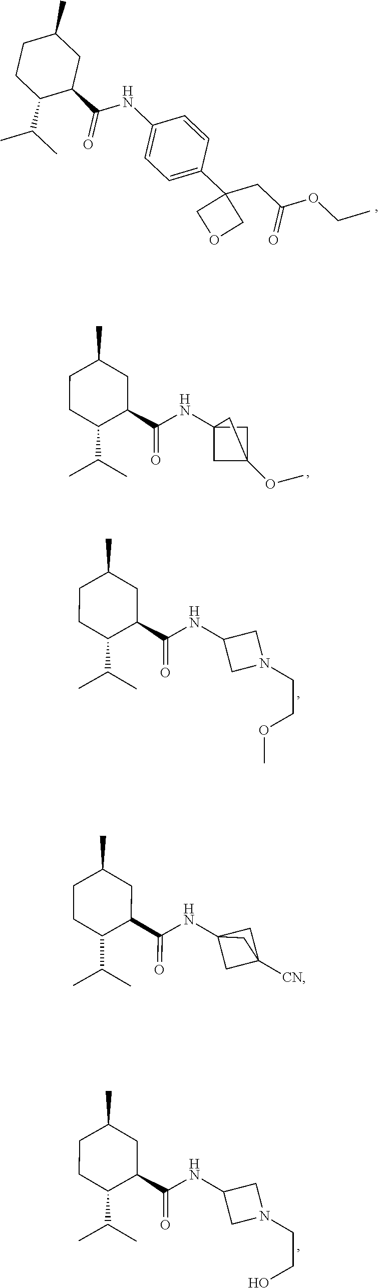 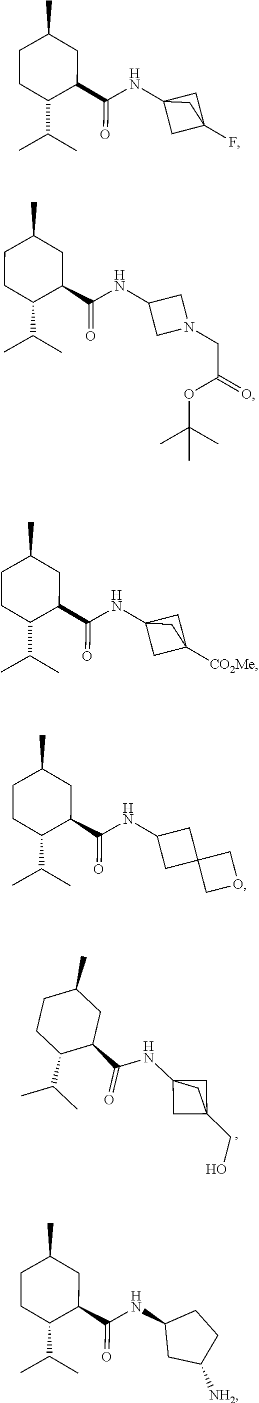 [SMILES: CC(C)[C@@H]1CC[C@@H](C)C[C@H]1C(=O)NC12CC(F)(C1)C2 ; CC(C)[C@@H]1CC[C@@H](C)C[C@H]1C(=O)NC1CN(CC(=O)OC(C)(C)C)C1 ; COC(=O)C12CC(NC(=O)[C@@H]3C[C@H](C)CC[C@H]3C(C)C)(C1)C2 ; CC(C)[C@@H]1CC[C@@H](C)C[C@H]1C(=O)NC1CC2(COC2)C1 ; CC(C)[C@@H]1CC[C@@H](C)C[C@H]1C(=O)NC12CC(CO)(C1)C2 ; CC(C)[C@@H]1CC[C@@H](C)C[C@H]1C(=O)N[C@H]1CC[C@H](N)C1] 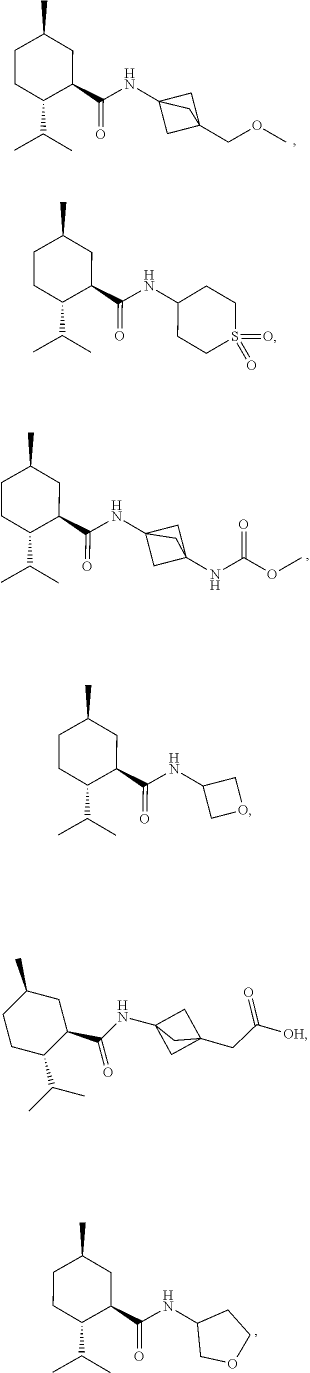 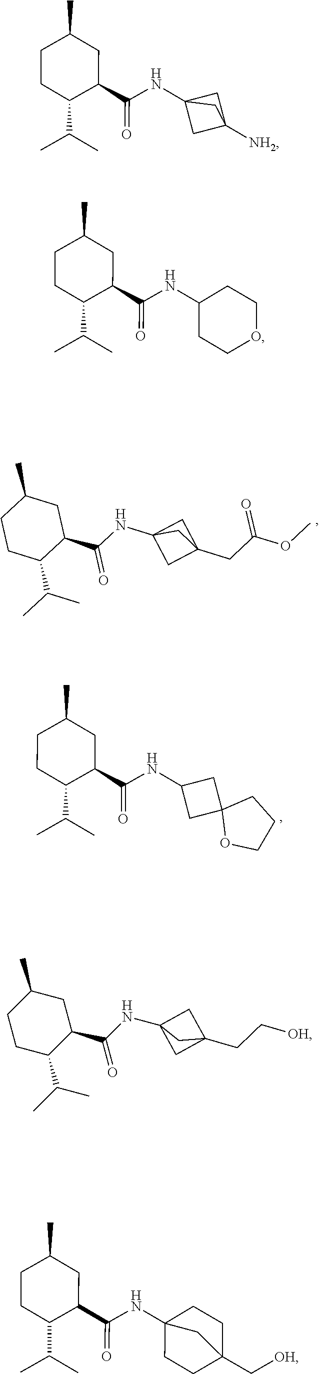 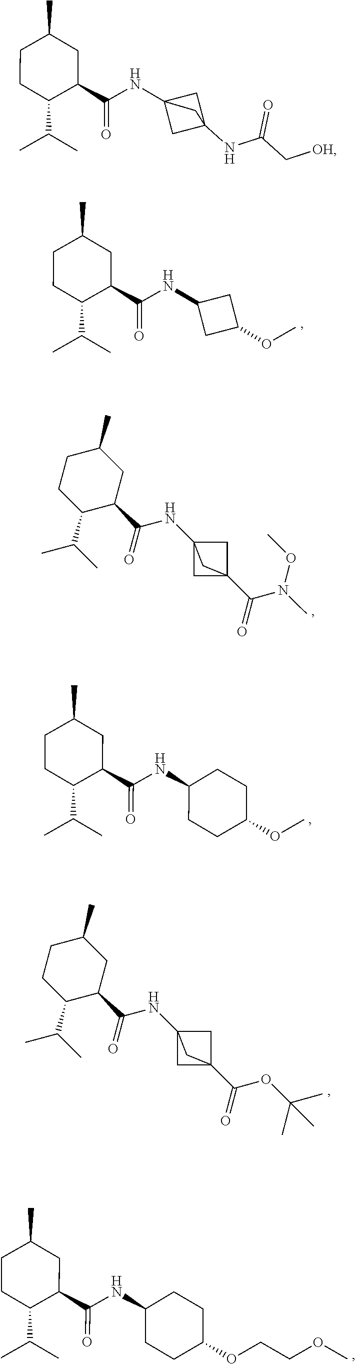 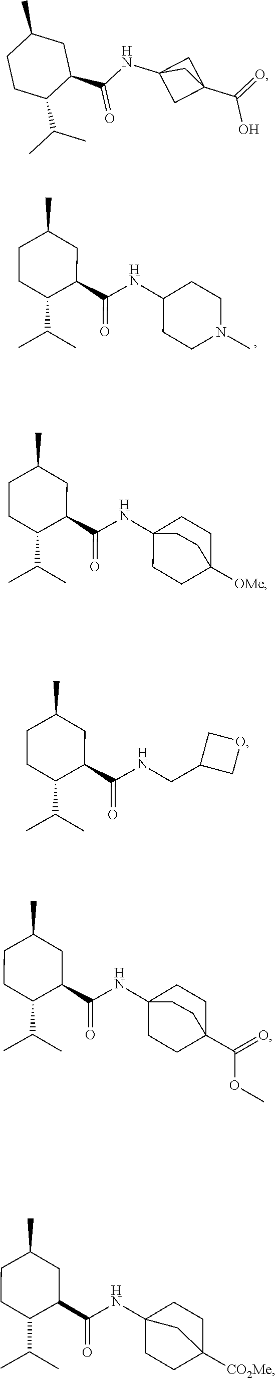 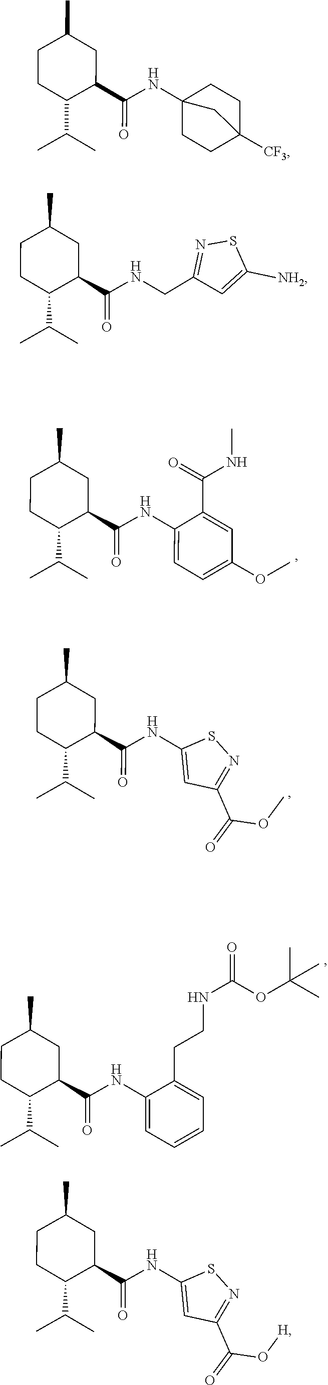 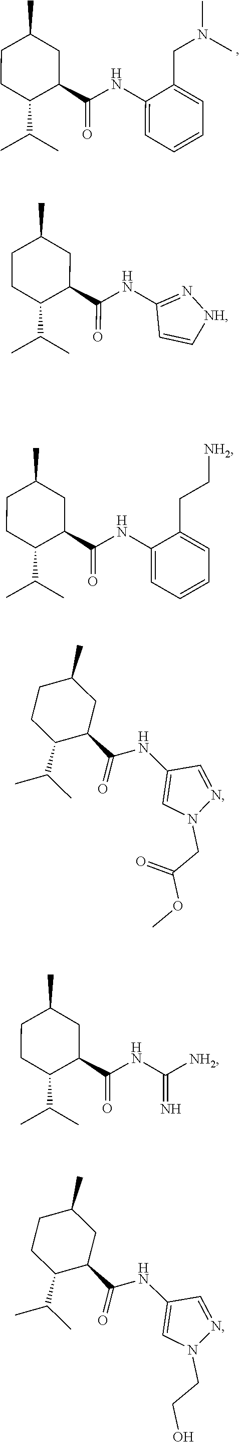 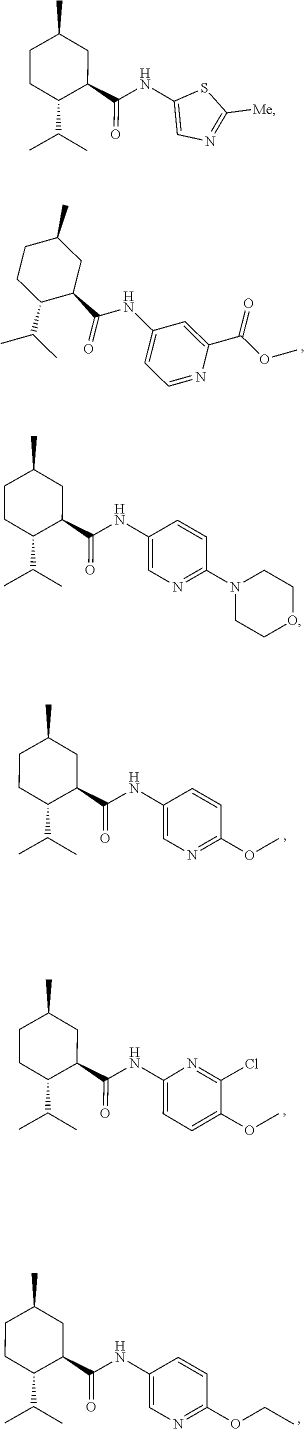 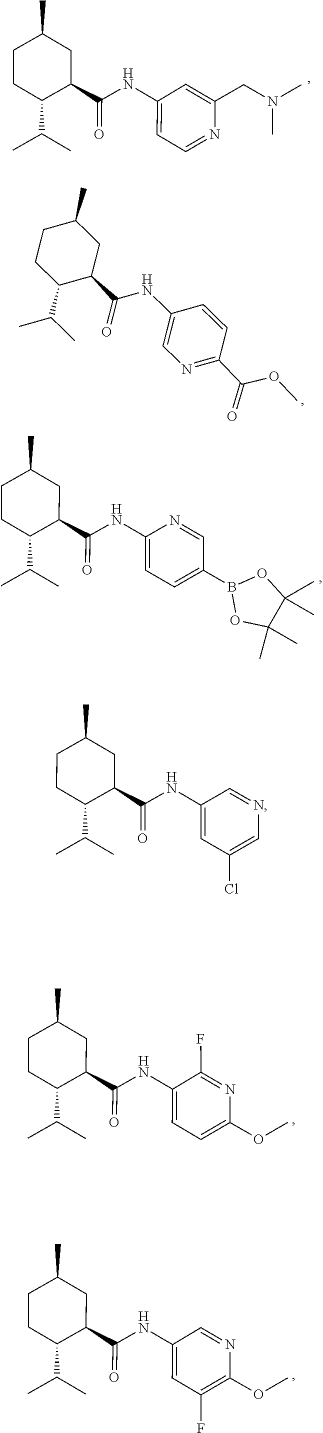 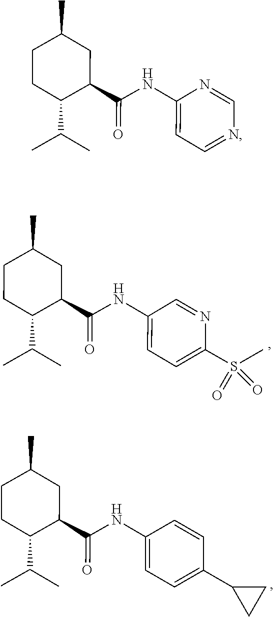 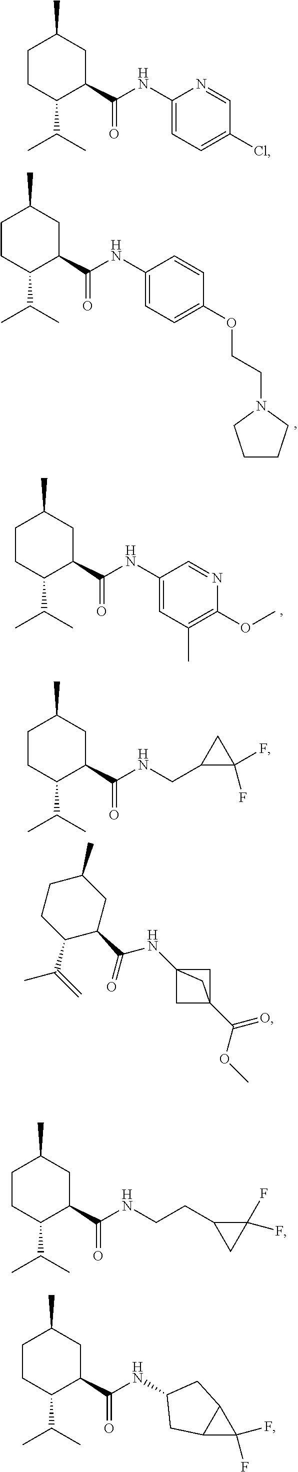 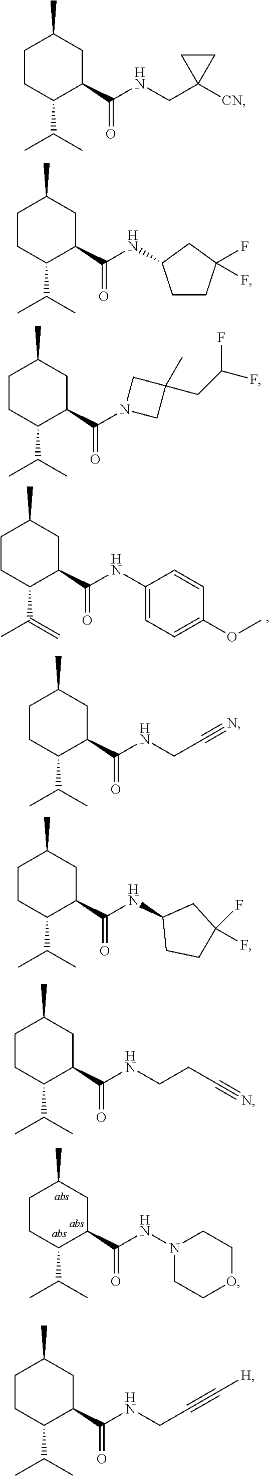 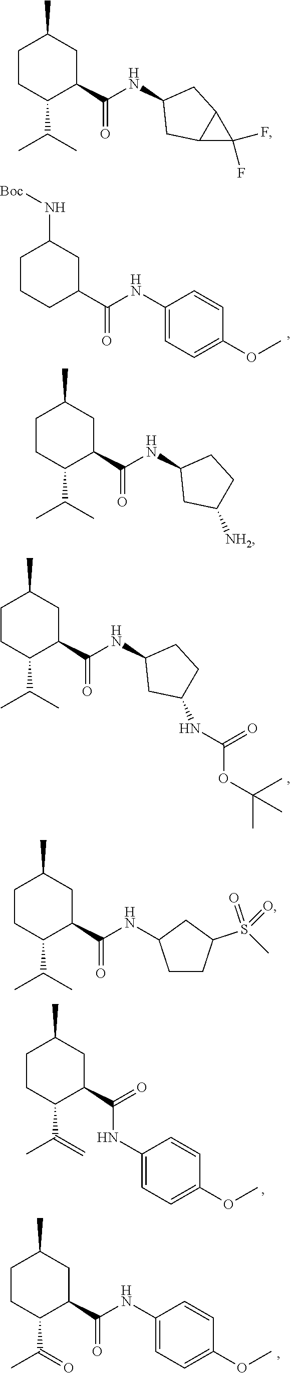 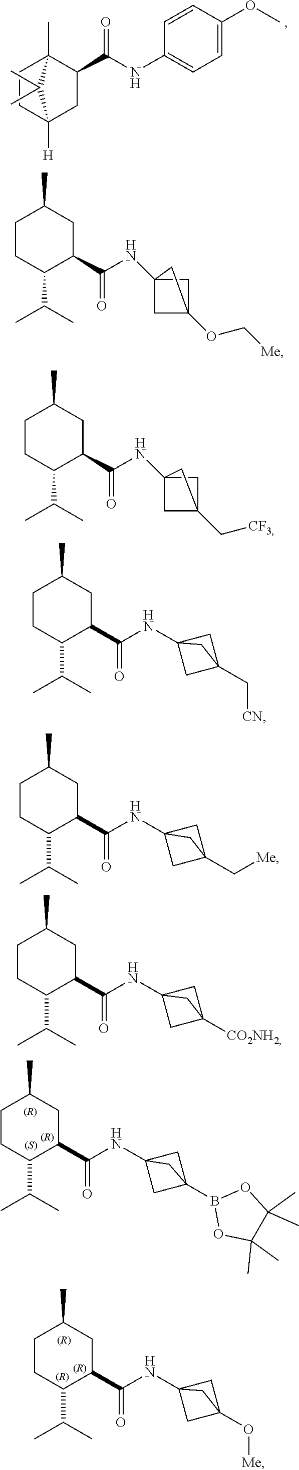 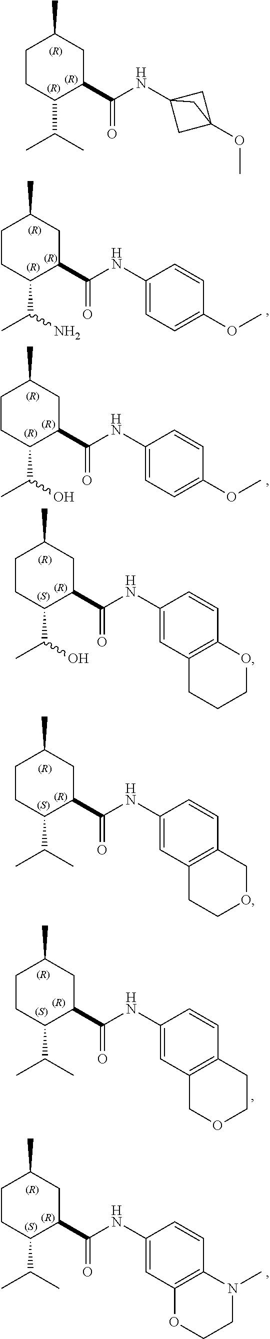 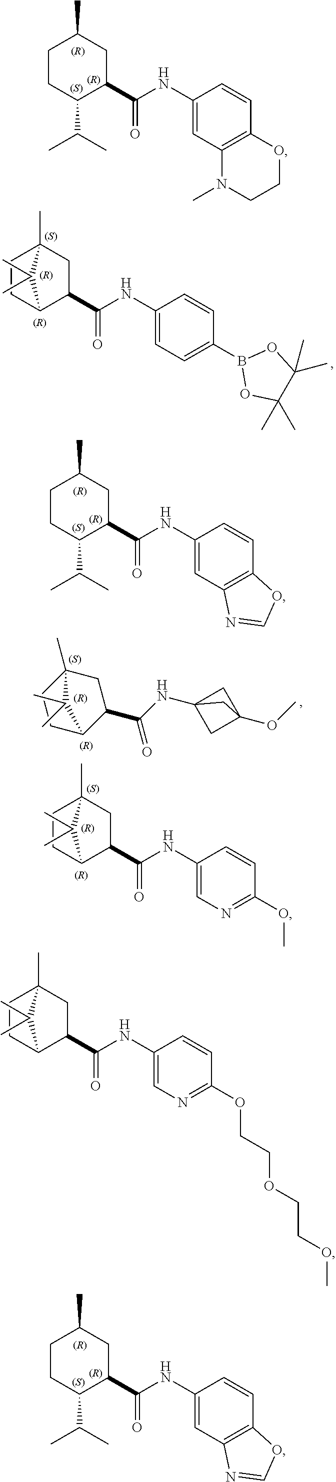 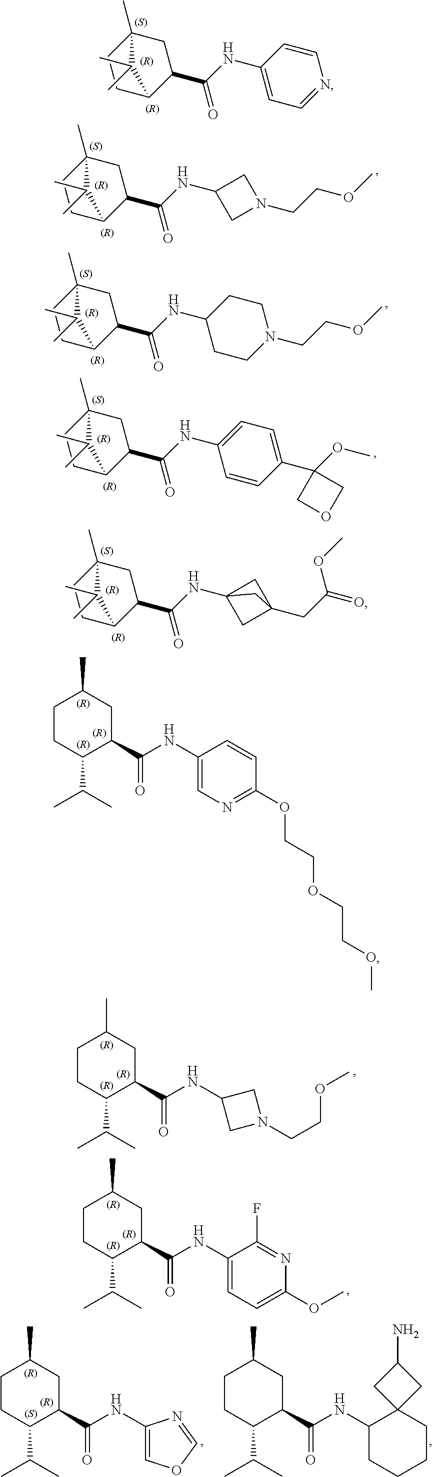 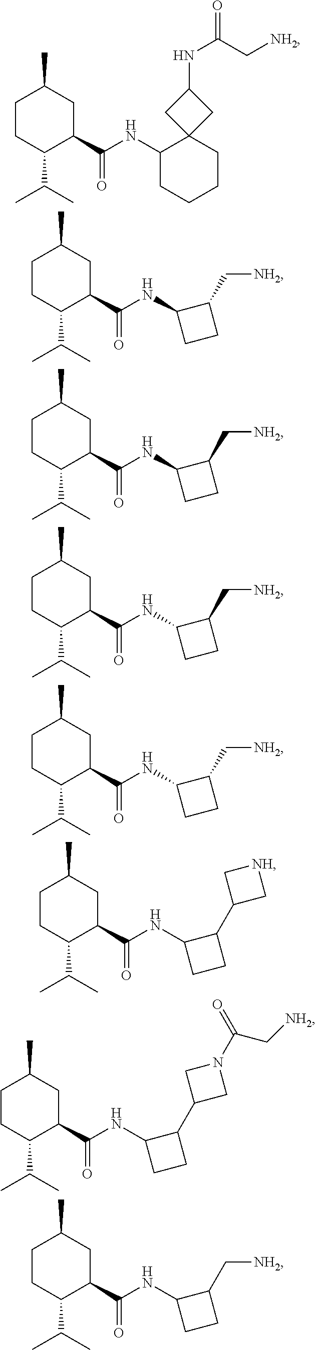 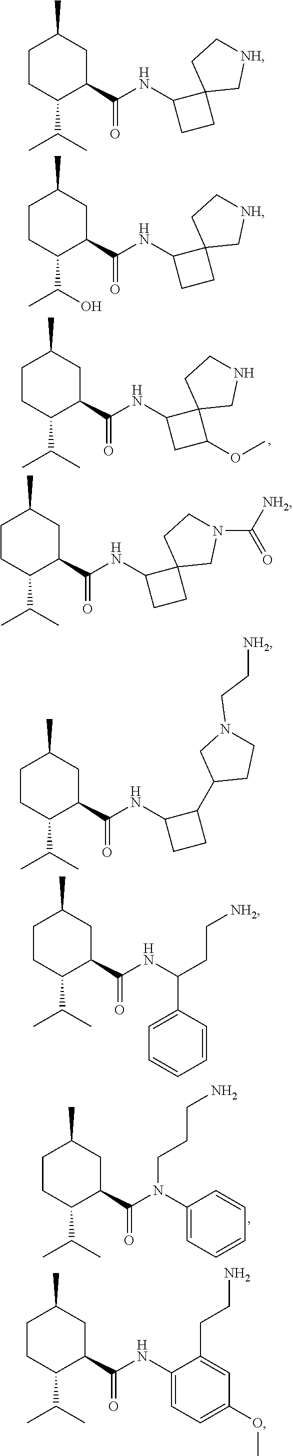 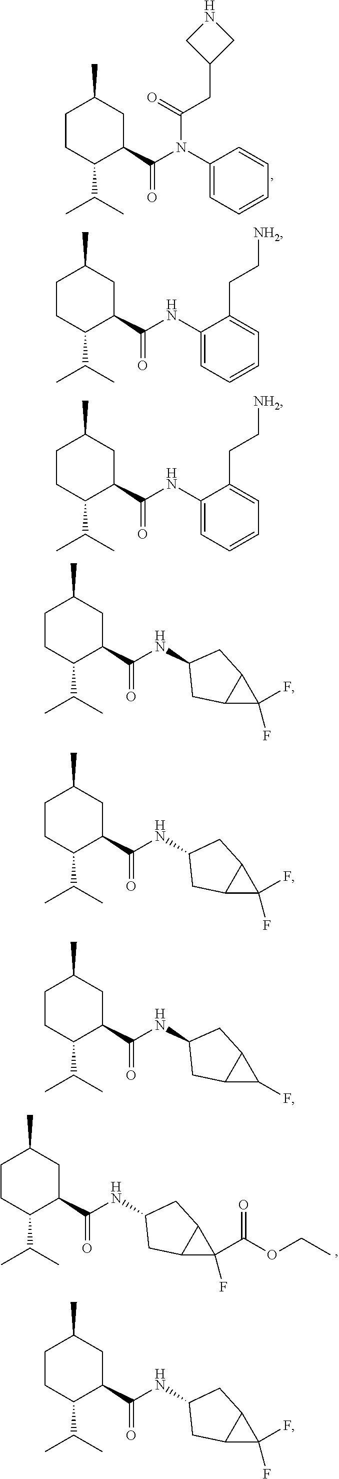 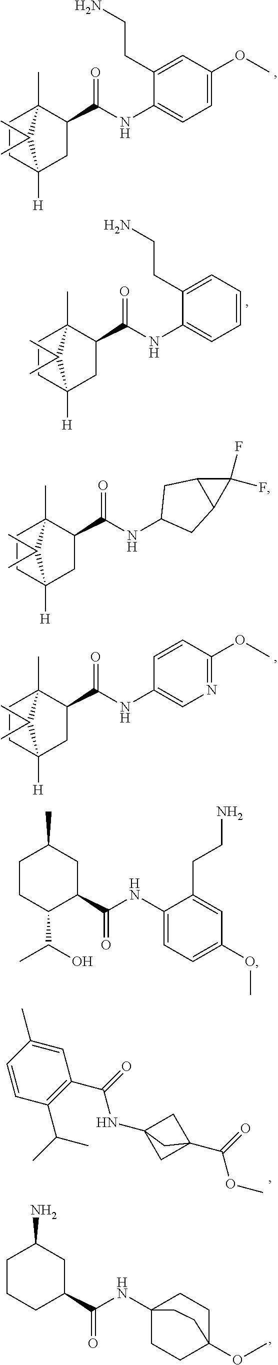 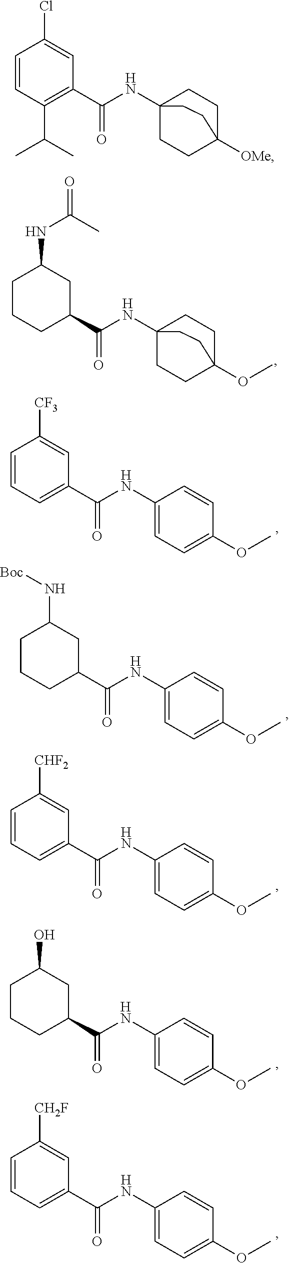
or a pharmaceutically acceptable salt thereof.

23. A process for preparing the compound of claim 1, comprising a condensation reaction that includes contacting 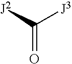
with 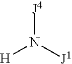
wherein
J³ is OH or Cl,
such that the compound, 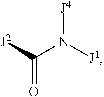
or a salt thereof, is formed,
wherein
J¹, J², and J⁴ are as defined in claim 1.

24. A composition, comprising the compound of claim 1.

25. A pharmaceutical composition, comprising the compound of claim 1 and a pharmaceutically acceptable carrier.

26. A method, comprising administration of the compound of claim 1 to a subject.

27. A method of activating an ion channel in an ocular tissue of a subject, comprising administration of the compound of claim 1 to the subject.

28. A method of modulating a transient receptor potential cation channel subfamily M member 8 (TRPM8) activity, comprising contacting the TRPM8 channel with the compound of claim 1.

29. A method of modulating an ion channel activity, comprising contacting the ion channel with the compound of claim 1.

30. A method of modulating a transient receptor potential (TRP) channel activity, comprising contacting the TRP channel with the compound of claim 1.

31. A method of treating an inflammatory eye disease, comprising administration of the compound of claim 1 to a subject in need thereof.

32. A method of treating uveitis, comprising administration of the compound of claim 1 to a subject in need thereof.

33. A method of treating a cardiovascular disease, comprising administration of the compound of claim 1 to a subject in need thereof.

34. A method of treating an inflammatory disease, comprising administration of the compound of claim 1 to a subject in need thereof.

35. A method of treating a cancer, comprising administration of the compound of claim 1 to a subject in need thereof.

36. A method of treatment, comprising administration of the compound of claim 1 to a subject in need thereof, wherein the treatment is selected from: treatment of eye disease; an anti-inflammatory treatment; treatment of skin disease; treatment of cardiovascular disease; treatment of auto-immune disorder; or treatment of disease characterized by abnormal growth.

37. A method of treatment, comprising administration of the compound of claim 1 to a subject in need thereof, wherein the treatment is selected from: treatment of non-infectious uveitis, non-infectious chorioretinitis, iritis, sterile conjunctivitis, keratitis, episcleritis, a dry eye disease, meibomian gland dysfunction, allergic conjunctivitis, glaucoma, or a retinal disease; treatment of rheumatoid arthritis, Crohn's disease, or ulcerative colitis; or treatment of prostate cancer.

38. The method of claim 32, wherein the administration is administration by topical instillation.

39. An article of manufacture, comprising the compound of claim 1, and instructions for use.
